(19) United States
(12) Patent Application Publication
(10) Pub. No.: US 2024/0034772 A1
(43) Pub. Date: Feb. 1, 2024

(54) ANTIBODIES BINDING TO F-PROTEIN OF METAPNEUMOVIRUS AND USES THEREOF

(71) Applicant: HUMABS BIOMED SA, Bellinzona (CH)

(72) Inventors: Fabio BENIGNI, Bellinzona (CH); Davide CORTI, Bellinzona (CH); Anna DEMARCO, Bellinzona (CH)

(21) Appl. No.: 18/256,419

(22) PCT Filed: Dec. 7, 2021
(86) PCT No.: PCT/EP2021/084520

§ 371 Date: Jun. 7, 2023

(30) Foreign Application Priority Data
Dec. 8, 2020 (WO) PCT/EP2020/085014
Dec. 11, 2020 (WO) PCT/EP2020/085747

(51) Int. Cl. C07K 16/10; A61P 31/14; G01N 33/569; C12N 15/63

(52) U.S. Cl. CPC C07K 16/1027; A61P 31/14; G01N 33/56983; C12N 15/63; C07K 2317/565; C07K 2317/52; C07K 2317/622

(57) ABSTRACT

The present invention relates to antibodies, and antigen binding fragments thereof, that bind to the F-protein (fusion protein) of metapneumovims (MPV). The antibodies, and antigen binding fragments thereof, neutralize infection of MPV. The invention also relates to nucleic acids that encode, and to cells that express such antibodies and antibody fragments. In addition, the invention relates to the use of the antibodies and antibody fragments in methods for detecting and checking an MPV antigen as well as in the diagnosis, treatment and prevention of MPV infection.

The present invention relates to the field of antibodies against metapneumovirus (MPV), in particular to antibodies binding to the F protein (fusion protein) of metapneumovirus (MPV) in its pre-fusion conformation. The present invention also relates to the use of such antibodies, e.g. in a method for neutralization of MPV infection, in a method for detecting MPV antigens or in a method for testing MPV vaccines.

The human metapneumovirus (hMPV) was isolated for the first time in 2001 and is a common cause of bronchiolitis and pneumonia among children and the elderly. MPV also causes repeated infections including severe lower respiratory tract disease, which may occur at any age, especially among the elderly or those with compromised cardiac, pulmonary, or immune systems. MPV is associated with 5% to 40% of respiratory tract infections in hospitalized and outpatient children. Infection with MPV is a significant burden of disease in at-risk premature infants, chronic lung disease of prematurity, congestive heart disease, and immunodeficiency (Martino et al., 2005, Biology of Blood and Marrow Transplantation: Journal of the American Society for Blood and Marrow Transplantation 11:781-796).

MPV, which belongs to the Metapneumovirus genus of the subfamily Pneumoviriniae and family Paramyxoviridae, is an enveloped non-segmented negative-strand RNA virus. The genetic structure of MPV is similar to that of respiratory syncytial virus (RSV), although MPV lacks the non-structural genes NS1 and NS2 found in RSV. The RSV and MPV envelopes contain three virally encoded transmembrane surface glycoproteins: the major attachment protein G, the fusion protein F, and the small hydrophobic SH protein.

The MPV F protein directs viral penetration by fusion between the virion envelope and the host cell plasma membrane. Initially, the MPV F protein is expressed as polypeptide precursor (“F₀”). The F-protein precursor F₀ is proteolytically processed at a conserved cleavage site, resulting in F₁ and F₂ polypeptides. The mature trimeric F protein is formed by assembly of three protomers of the F₂-F₁ heterodimer and adopts a metastable pre-fusion conformation. The N-terminus of the F subunit that is created by proteolytic cleavage and contains hydrophobic stretch of amino acids, called the fusion peptide, can insert directly into the target membrane to initiate fusion. After binding to the target cell and subsequent activation, the metastable pre-fusion F protein undergoes a series of structural rearrangements that result in the fusion of viral and target-cell membranes and in the formation of the stable post-fusion F protein.

The MPV F protein is the major target of neutralizing antibodies against MPV and the subject of vaccine development. However, despite the identification of potently neutralizing antibodies, such as MPE8 (Corti etal., 2013, Cross-neutralization of four paramyxoviruses by a human monoclonal antibody. Nature 501: 439-443), there is still a need for antibodies showing increased affinity to MPV pre-fusion F protein in order to provide potent MPV neutralization, for detection of MPV antigens, e.g. in diagnosis, and for the development of vaccines against MPV.

In view of the above, it is the object of the present invention to overcome the drawbacks of the prior art. In particular, it is an object of the present invention to provide antibodies, which bind to the pre-fusion F-protein of MPV with high binding affinity. It is also an object of the present invention to provide antibodies, which potently neutralize MPV infection.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%, for example, x±5%, or x±7%, or x±10%, or x±12%, or x±15%, or x±20%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits. In some embodiments, the patient is a human.

Doses are often expressed in relation to the bodyweight. Thus, a dose which is expressed as [g, mg, or other unit]/kg (or g, mg etc.) usually refers to [g, mg, or other unit] “per kg (or g, mg etc.) bodyweight”, even if the term “bodyweight” is not explicitly mentioned.

The term “binding” and similar reference usually means “specifically binding”, which does not encompass non-specific sticking.

As used herein, the term “antibody” encompasses various forms of antibodies including, without being limited to, whole antibodies, antibody fragments (such as antigen binding fragments), human antibodies, chimeric antibodies, humanized antibodies, recombinant antibodies and genetically engineered antibodies (variant or mutant antibodies) as long as the characteristic properties according to the invention are retained. In some embodiments, the antibody is a human antibody. In some embodiments, the antibody is a monoclonal antibody. For example, the antibody is a human monoclonal antibody.

As described above, the term “antibody” generally also includes antibody fragments. Fragments of the antibodies may retain the antigen-binding activity of the antibodies. Such fragments are referred to as “antigen-binding fragments”. Antigen-binding fragments include, but are not limited to, single chain antibodies, Fab, Fab′, F(ab′)2, Fv or scFv. Fragments of the antibodies can be obtained from the antibodies by methods that include digestion with enzymes, such as pepsin or papain, and/or by cleavage of disulfide bonds by chemical reduction. Alternatively, fragments of the antibodies can be obtained by recombinant means, for example by cloning and expressing a part (fragment) of the sequences of the heavy and/or light chain. The invention also encompasses single-chain Fv fragments (scFv) derived from the heavy and light chains of an antibody of the invention. For example, the invention includes a scFv comprising the CDRs from an antibody of the invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker. Antibody fragments of the invention may be contained in a variety of structures known to the person skilled in the art. In addition, the sequences of the invention may be a component of multispecific molecules in which the sequences of the invention target the epitopes of the invention and other regions of the molecule bind to other targets. Although the specification, including the claims, may, in some places, refer explicitly to antigen binding fragment(s), antibody fragment(s), variant(s) and/or derivative(s) of antibodies, it is understood that the term “antibody” includes all categories of antibodies, namely, antigen binding fragment(s), antibody fragment(s), variant(s) and derivative(s) of antibodies.

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). In some embodiments, human monoclonal antibodies are prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5. As used herein, the term “variable region” (variable region of a light chain (V_{L}), variable region of a heavy chain (V_{H})) denotes each of the pair of light and heavy chains which is involved directly in binding the antibody to the antigen.

Antibodies of the invention can be of any isotype (e.g., IgA, IgG, IgM i.e. an α, γ or μ heavy chain). For example, the antibody is of the IgG type. Within the IgG isotype, antibodies may be IgG1, IgG2, IgG3 or IgG4 subclass, for example IgG1. Antibodies of the invention may have a κ or a λ light chain. In some embodiments, the antibody is of IgG1 type and has a κ light chain.

Antibodies according to the present invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies according to the present invention may be immunogenic in human and/or in non-human (or heterologous) hosts e.g., in mice. For example, the antibodies may have an idiotope that is immunogenic in non-human hosts, but not in a human host. Antibodies of the invention for human use include those that cannot be easily isolated from hosts such as mice, goats, rabbits, rats, non-primate mammals, etc. and cannot generally be obtained by humanization or from xeno-mice.

As used herein, a “neutralizing antibody” is one that can neutralize, i.e., prevent, inhibit, reduce, impede or interfere with, the ability of a pathogen to initiate and/or perpetuate an infection in a host. The terms “neutralizing antibody” and “an antibody that neutralizes” or “antibodies that neutralize” are used interchangeably herein. These antibodies can be used alone, or in combination, as prophylactic or therapeutic agents upon appropriate formulation, in association with active vaccination, as a diagnostic tool, or as a production tool as described herein.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### Antibodies and Antigen-Binding Fragments Thereof

In a first aspect the present invention provides an (isolated) antibody, or an antigen-binding fragment thereof, which (specifically) binds to the F-protein (fusion protein) of metapneumovirus (MPV), in particular of human metapneumovirus (hMPV).

The MPV F protein is a type I trans-membrane surface protein that has an N-terminal cleaved signal peptide and a membrane anchor near the C-terminus. The MPV F protein is synthesized as inactive F0 precursor that assembles into a homotrimer and is activated by cleavage. The F protein is formed by three domains (DI to DIII), a fusion peptide (FP) and three heptad-repeats regions (HR-A, -B and -C). The MPV F glycoprotein directs viral penetration by fusion between the virion envelope and the host cell plasma membrane. The N-terminus of the F subunit, that is created by proteolytic cleavage and contains the fusion peptide, inserts directly into the target membrane to initiate fusion. After binding to the target cell and subsequent activation, the metastable pre-fusion F protein undergoes a series of structural rearrangements that result in the insertion of the fusion peptide into the target cell membrane, followed by the formation of a stable helical bundle that forms as the viral and cell membranes are apposed. These structural changes lead to the formation of a stable post-fusion F protein. Later in infection, the F protein expressed on the cell surface of infected cells can mediate fusion with adjacent non-infected cells forming large syncytia.

In some embodiments, the antibody or the antigen-binding fragment thereof binds to the pre-fusion F protein of MPV, in particular of human metapneumovirus (hMPV).

The pre-fusion F protein is the relevant conformation to block virus entry. Accordingly, antibodies recognizing the pre-fusion conformation of the MPV F protein are particularly effective in neutralization.

In some embodiments, the binding affinity of the antibody, or the antigen-binding fragment thereof, is higher for the pre-fusion F protein than for the post-fusion F protein. Accordingly, the antibody, or the antigen-binding fragment thereof, may be selective for pre-fusion F protein (over post-fusion F protein). Recognition of the abundant post-fusion F protein, that can act as a decoy, consumes the antibody, thereby reducing its efficacy, which is preferably reduced or avoided.

More specifically, an at least 100 fold higher concentration of the antibody, or the antigen-binding fragment thereof, may be required for 50% antibody binding to the post-fusion F protein of MPV than for 50% antibody binding to the pre-fusion F protein of MPV.

Standard methods to assess binding of the antibody according to the present invention, or the antigen-binding fragment thereof, are known to those skilled in the art and include, for example, ELISA (enzyme-linked immunosorbent assay). Thereby, the relative affinities of antibody binding may be determined by measuring the concentration of the antibody (EC₅₀) required to achieve 50% maximal binding at saturation.

An exemplary standard ELISA may be performed as follows: ELISA plates may be coated with a sufficient amount (e.g., 1 μg/ml) of the protein/complex/particle to which binding of the antibody is to be tested (e.g., MPV F protein in pre-fusion or post-fusion conformation). Plates may then be incubated with the antibody to be tested. After washing, antibody binding can be revealed, e.g. using a labelled antibody recognizing the test antibody, such as goat anti-human IgG coupled to alkaline phosphatase. Plates may then be washed, the required substrate (e.g., p-NPP) may be added and plates may be read, e.g. at 405 nm. The relative affinities of antibody binding may be determined by measuring the concentration of mAb (EC₅₀) required to achieve 50% maximal binding at saturation. The EC₅₀ values may be calculated by interpolation of binding curves fitted with a four-parameter nonlinear regression with a variable slope.

In some embodiments, the antibody, or the antigen-binding fragment thereof, neutralizes infection of MPV, in particular hMPV. The antibody and antigen binding fragment of the invention may have high neutralizing potency. The concentration of the antibody of the invention required for 50% neutralization of MPV, is, for example, about 500 ng/ml or less. In certain embodiments, the concentration of the antibody of the invention required for 50% neutralization of MPV is about 500, 450, 400, 350, 300, 250, 200, 175, 150, 125 or about 100 ng/ml or less. This means that only low concentrations of antibody are required for 50% neutralization of MPV. Specificity and potency can be measured using standard assays as known to one of skill in the art.

For example, to study and quantitate neutralization in the laboratory the person skilled in the art knows various standard “neutralization assays”. For a neutralization assay, the viruses (to be neutralized) are typically propagated in cells and/or cell lines. For example, in a neutralization assay cultured cells may be incubated with a fixed amount of MPV (hMPV) in the presence (or absence) of the antibody to be tested. As a readout for example flow cytometry may be used. Alternatively, also other readouts are conceivable.

In some embodiments, the antibody or the antigen-binding fragment thereof binds specifically to F-proteins of MPV subgroups A1, A2, B1, and B2, i.e. to all four subgroups of MPV. Accordingly, the antibody or the antigen-binding fragment thereof may neutralize infection of MPV subgroups A1, A2, B1, and B2, i.e. to all four subgroups of MPV. The amino acid sequences of the G and F proteins of MPV are classified into A and B groups and further divided in 4 subgroups: A1, A2, B1 and B2. As shown in the appended examples, the antibodies or antigen binding fragments of the invention bind specifically to all four subgroups of MPV: A1, A2, B1, and B2. Moreover, in some embodiments, the antibodies or antigen binding fragments of the invention potently neutralize all four subgroups of MPV: A1, A2, B1, and B2.

In some embodiments, the antibody, or the antigen-binding fragment thereof, binds to the same (or an overlapping) epitope as MPE8 (e.g., as described in Corti et al., 2013, Cross-neutralization of four paramyxoviruses by a human monoclonal antibody. Nature 501: 439-443, which is incorporated herein by reference). MPE8 was described to bind to an epitope near the midsection of the RSV (and likely MPV) F ectodomain at the intersection of DI, DII and DIII domains from two subunits of the F trimer (also referred to “antigenic site III”).

In other embodiments, the antibody, or the antigen-binding fragment thereof, binds to an epitope distinct from (and non-overlapping with) the epitope of MPE8 on the MPV F-protein (i.e., distinct from antigenic site III).

In general, the antibody, or an antigen-binding fragment thereof, according to the present invention, may comprise (at least) three complementarity determining regions (CDRs) on a heavy chain and (at least) three CDRs on a light chain. In general, complementarity determining regions (CDRs) are the hypervariable regions present in heavy chain variable domains and light chain variable domains. Typically, the CDRs of a heavy chain and the connected light chain of an antibody together form the antigen receptor. Usually, the three CDRs (CDR1, CDR2, and CDR3) are arranged non-consecutively in the variable domain. Since antigen receptors are typically composed of two variable domains (on two different polypeptide chains, i.e. heavy and light chain: heavy chain variable region (VH) and light chain variable region (VL)), there are typically six CDRs for each antigen receptor (heavy chain: CDRH1, CDRH2, and CDRH3; light chain: CDRL1, CDRL2, and CDRL3). For example, a classical IgG antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a variable region (or each variable region, respectively) may be composed of four framework regions, separated by three CDR's.

The sequences of the heavy chains and light chains of exemplary antibodies of the invention, comprising three different CDRs on the heavy chain and three different CDRs on the light chain were determined. The position of the CDR amino acids are defined according to the IMGT numbering system (IMGT: http://www.imgt.org/; cf. Lefranc, M.-P. et al. (2009) Nucleic Acids Res. 37, D1006-D1012).

In some embodiments, the antibody, or an antigen-binding fragment thereof, comprises (i) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 6, and SEQ ID NO: 7, respectively; or (iii) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 16, and SEQ ID NO: 18, respectively; or (iv) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 17, and SEQ ID NO: 18, respectively.

In some embodiments, the antibody, or an antigen-binding fragment thereof, comprises (i) heavy chain CDR1, CDR2, and CDR3 sequences having at least 80% sequence identity with the amino acid sequences of SEQ ID NO: 1, SEQ ID NO: 2, and SEQ ID NO: 3, respectively, and light chain CDR1, CDR2, and CDR3 sequences having at least 80% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 80% sequence identity with the amino acid sequences of SEQ ID NO: 1, SEQ ID NO: 2, and SEQ ID NO: 3, respectively, and light chain CDR1, CDR2, and CDR3 sequences having at least 80% sequence identity with the amino acid sequences of SEQ ID NO: 4, SEQ ID NO: 6, and SEQ ID NO: 7, respectively; or (iii) heavy chain CDR1, CDR2, and CDR3 sequences having at least 80% sequence identity with the amino acid sequences of SEQ ID NO: 12, SEQ ID NO: 13, and SEQ ID NO: 14, respectively, and light chain CDR1, CDR2, and CDR3 sequences having at least 80% sequence identity with the amino acid sequences of SEQ ID NO: 15, SEQ ID NO: 16, and SEQ ID NO: 18, respectively; or (iv) heavy chain CDR1, CDR2, and CDR3 sequences having at least 80% sequence identity with the amino acid sequences of SEQ ID NO: 12, SEQ ID NO: 13, and SEQ ID NO: 14, respectively, and light chain CDR1, CDR2, and CDR3 sequences having at least 80% sequence identity with the amino acid sequences of SEQ ID NO: 15, SEQ ID NO: 17, and SEQ ID NO: 18, respectively.

As used throughout the present specification, “sequence identity” is usually calculated with regard to the full length of the reference sequence (i.e. the sequence recited in the application). Percentage identity, as referred to herein, can be determined, for example, by methods known in the art, such as BLAST using the default parameters specified by the NCBI (the National Center for Biotechnology Information; http://www.ncbi.nlm.nih.gov/) [Blosum 62 matrix; gap open penalty=11 and gap extension penalty=1].

An “sequence variant” has an altered sequence in which one or more of the amino acids in the reference sequence is/are deleted or substituted, and/or one or more amino acids is/are inserted into the sequence of the reference amino acid sequence. As a result of the alterations, the amino acid sequence variant has an amino acid sequence which is at least 70% identical to the reference sequence. Variant sequences which are at least 70% identical have no more than 30 alterations, i.e. any combination of deletions, insertions or substitutions, per 100 amino acids of the reference sequence. In a “sequence variant” the functionality of the reference sequence (e.g., in the present case binding to the F protein of MPV) may be maintained.

In general, while it is possible to have non-conservative amino acid substitutions, the substitutions are usually conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, cysteine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

The antibody, or an antigen-binding fragment thereof, of the present invention may comprise (i) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 1, SEQ ID NO: 2, and SEQ ID NO: 3, respectively, and light chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 4, SEQ ID NO: 5, and SEQ ID NO: 7, respectively; or (ii) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 1, SEQ ID NO: 2, and SEQ ID NO: 3, respectively, and light chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 4, SEQ ID NO: 6, and SEQ ID NO: 7, respectively; or (iii) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 12, SEQ ID NO: 13, and SEQ ID NO: 14, respectively, and light chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 15, SEQ ID NO: 16, and SEQ ID NO: 18, respectively; or (iv) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 12, SEQ ID NO: 13, and SEQ ID NO: 14, respectively, and light chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 15, SEQ ID NO: 17, and SEQ ID NO: 18, respectively.

In some embodiments, the antibody or the antigen-binding fragment thereof comprises:
a heavy chain CDR1 sequence having at least 95% sequence identity with the amino acid sequences of SEQ ID NO: 1;
a heavy chain CDR2 sequence having at least 95% sequence identity with the amino acid sequences of SEQ ID NO: 2;
a heavy chain CDR3 sequence having at least 85% or 90% sequence identity with the amino acid sequences of SEQ ID NO: 3;
a light chain CDR1 sequence having at least 95% sequence identity with the amino acid sequences of SEQ ID NO: 4;
a light chain CDR2 sequence having at least 95% sequence identity with the amino acid sequences of SEQ ID NO: 5 or 6; and
a light chain CDR3 sequence having at least 95% sequence identity with the amino acid sequences of SEQ ID NO: 7.

More specifically, the antibody or the antigen-binding fragment thereof may comprise:
a heavy chain CDR1 sequence according to SEQ ID NO: 1;
a heavy chain CDR2 sequence according to SEQ ID NO: 2;
a heavy chain CDR3 sequence having at least 90% sequence identity with the amino acid sequences of SEQ ID NO: 3;
a light chain CDR1 sequence according to SEQ ID NO: 4;
a light chain CDR2 sequence according to SEQ ID NO: 5 or 6; and
a light chain CDR3 sequence according to SEQ ID NO: 7.

In some embodiments, the C-terminal Asp (D) residue in SEQ ID NO: 3 is substituted. More specifically, the C-terminal Asp residue in SEQ ID NO: 3 may be substituted with another polar amino acid. Examples of polar amino acids include arginine, asparagine, aspartic acid, glutamine, glutamic acid, histidine, lysine, serine, threonine and tyrosine. For example, the C-terminal aspartic acid residue in SEQ ID NO: 3 may be substituted with a histidine residue. Accordingly, the antibody or the antigen-binding fragment thereof may comprise a heavy chain CDR3 sequence according to SEQ ID NO: 3 or 10.

Accordingly, the antibody or the antigen-binding fragment thereof may comprise:
a heavy chain CDR1 sequence according to SEQ ID NO: 1;
a heavy chain CDR2 sequence according to SEQ ID NO: 2;
a heavy chain CDR3 sequence according to SEQ ID NO: 3;
a light chain CDR1 sequence according to SEQ ID NO: 4;
a light chain CDR2 sequence according to SEQ ID NO: 5 or 6; and
a light chain CDR3 sequence according to SEQ ID NO: 7.

Alternatively, the antibody or the antigen-binding fragment thereof may comprise:
a heavy chain CDR1 sequence according to SEQ ID NO: 1;
a heavy chain CDR2 sequence according to SEQ ID NO: 2;
a heavy chain CDR3 sequence according to SEQ ID NO: 10;
a light chain CDR1 sequence according to SEQ ID NO: 4;
a light chain CDR2 sequence according to SEQ ID NO: 5 or 6; and
a light chain CDR3 sequence according to SEQ ID NO: 7.

In other embodiments, the antibody or the antigen-binding fragment thereof comprises:
a heavy chain CDR1 sequence having at least 70% sequence identity with the amino acid sequences of SEQ ID NO: 12;
a heavy chain CDR2 sequence having at least 95% sequence identity with the amino acid sequences of SEQ ID NO: 13;
a heavy chain CDR3 sequence having at least 95% sequence identity with the amino acid sequences of SEQ ID NO: 14;
a light chain CDR1 sequence having at least 95% sequence identity with the amino acid sequences of SEQ ID NO: 15;
a light chain CDR2 sequence having at least 95% sequence identity with the amino acid sequences of SEQ ID NO: 16 or 17; and
a light chain CDR3 sequence having at least 95% sequence identity with the amino acid sequences of SEQ ID NO: 18.

More specifically, the antibody or the antigen-binding fragment thereof may comprise:
a heavy chain CDR1 sequence having at least 70% sequence identity with the amino acid sequences of SEQ ID NO: 12;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

In some embodiments, one or more of the heavy chain variable region amino acid residues N34, S36 and C38 (corresponding to N6, S8 and C10, respectively, in SEQ ID NO: 12) is/are substituted. More specifically,
N34 (corresponding to N6 in SEQ ID NO: 12) may be substituted with another polar amino acid, such as Gln (Q) or Ser (S);
S36 (corresponding to S8 in SEQ ID NO: 12) may be substituted with another small amino acid, such as Ala (A); and/or
C38 (corresponding to C10 in SEQ ID NO: 12) may be substituted with any amino acid, such as Ser (S), Ala (A) or Tyr (Y).

Examples of polar amino acids include arginine, asparagine, aspartic acid, glutamine, glutamic acid, histidine, lysine, serine, threonine and tyrosine. Examples of small amino acids include alanine, serine, threonine, glycine, cysteine, proline, asparagine and aspartic acid with alanine, glycine and serine being particularly small.

The heavy chain CDR1 sequence the antibody or the antigen-binding fragment thereof may differ in up to three amino acids from SEQ ID NO: 12, in particular as described above. In some embodiments, the heavy chain CDR1 sequence the antibody or the antigen-binding fragment thereof differs in a single amino acid substitution from SEQ ID NO: 12, which may be selected from N34, S36 and C38, as described above. In other embodiments, the heavy chain CDR1 sequence the antibody or the antigen-binding fragment thereof differs in (exactly) two amino acid substitutions from SEQ ID NO: 12, which may be selected from N34/S36, N34/C38 and 536/C38, as described above, for example the amino acid residues at N34/C38 may be substituted as described above.

For example, the antibody or the antigen-binding fragment thereof may comprise a heavy chain CDR1 sequence according to any one of SEQ ID NOs 12, 21, 23, 25, 27, 29, 31 and 33.

Accordingly, the antibody or the antigen-binding fragment thereof may comprise:
a heavy chain CDR1 sequence according to SEQ ID NO: 12;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

Alternatively, the antibody or the antigen-binding fragment thereof may comprise:
a heavy chain CDR1 sequence according to SEQ ID NO: 21;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

Alternatively, the antibody or the antigen-binding fragment thereof may comprise:
a heavy chain CDR1 sequence according to SEQ ID NO: 23;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

Alternatively, the antibody or the antigen-binding fragment thereof may comprise:
a heavy chain CDR1 sequence according to SEQ ID NO: 25;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

Alternatively, the antibody or the antigen-binding fragment thereof may comprise:
a heavy chain CDR1 sequence according to SEQ ID NO: 27;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

Alternatively, the antibody or the antigen-binding fragment thereof may comprise:
a heavy chain CDR1 sequence according to SEQ ID NO: 29;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

Alternatively, the antibody or the antigen-binding fragment thereof may comprise:
a heavy chain CDR1 sequence according to SEQ ID NO: 31;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

Alternatively, the antibody or the antigen-binding fragment thereof may comprise:
a heavy chain CDR1 sequence according to SEQ ID NO: 33;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

In some embodiments, the antibody of the invention, or the antigen-binding fragment thereof, comprises (i) a heavy chain variable region (VH) comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8 and a light chain variable region (VL) comprising the amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3 or 10, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 19 and a light chain variable region comprising the amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, 21, 23, 25, 27, 29, 31 or 33; SEQ ID NO: 13; and SEQ ID NO: 14, respectively; and light chain CDR1, CDR2, and CDR3 sequences as set forth in SEQ ID NO: 15, SEQ ID NO: 16 or 17, and SEQ ID NO: 20, respectively) may be maintained.

In some embodiments, the antibody of the invention, or the antigen-binding fragment thereof, comprises (i) a heavy chain variable region comprising an amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8 and a light chain variable region comprising the amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3 or 10, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 19 and a light chain variable region comprising the amino acid sequence having 75% or more (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, 21, 23, 25, 27, 29, 31 or 33; SEQ ID NO: 13; and SEQ ID NO: 14, respectively; and light chain CDR1, CDR2, and CDR3 sequences as set forth in SEQ ID NO: 15, SEQ ID NO: 16 or 17, and SEQ ID NO: 20, respectively) may be maintained.

In some embodiments, the antibody of the invention, or the antigen-binding fragment thereof, comprises (i) a heavy chain variable region comprising an amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8 and a light chain variable region comprising the amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3 or 10, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 19 and a light chain variable region comprising the amino acid sequence having 80% or more (e.g., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, 21, 23, 25, 27, 29, 31 or 33; SEQ ID NO: 13; and SEQ ID NO: 14, respectively; and light chain CDR1, CDR2, and CDR3 sequences as set forth in SEQ ID NO: 15, SEQ ID NO: 16 or 17, and SEQ ID NO: 20, respectively) may be maintained.

In some embodiments, the antibody of the invention, or the antigen-binding fragment thereof, comprises (i) a heavy chain variable region comprising an amino acid sequence having 85% or more (e.g., 86%, 87%, 88%, 89%, 90%, 91%, 92%, 93%, 94%, 95%, 96%, 97%, 98%, 99% or more) identity to SEQ ID NO: 8 and a light chain variable region comprising the amino acid sequence having 85% or more (e.g., 86%, 87%, 88%, 89%, 90%, 91%, 92%, 93%, 94%, 95%, 96%, 97%, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3 or 10, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 85% or more (e.g., 86%, 87%, 88%, 89%, 90%, 91%, 92%, 93%, 94%, 95%, 96%, 97/s, 98%, 99% or more) identity to SEQ ID NO: 19 and a light chain variable region comprising the amino acid sequence having 85% or more (e.g., 86%, 87%, 88%, 89%, 90%, 91%, 92%, 93%, 94%, 95%, 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, 21, 23, 25, 27, 29, 31 or 33; SEQ ID NO: 13; and SEQ ID NO: 14, respectively; and light chain CDR1, CDR2, and CDR3 sequences as set forth in SEQ ID NO: 15, SEQ ID NO: 16 or 17, and SEQ ID NO: 20, respectively) may be maintained.

In some embodiments, the antibody of the invention, or the antigen-binding fragment thereof, comprises (i) a heavy chain variable region comprising an amino acid sequence having 90% or more (e.g., 91%, 92%, 93%, 94%, 95 0 /0, 96%, 97%, 98%, 99% or more) identity to SEQ ID NO: 8 and a light chain variable region comprising the amino acid sequence having 90% or more (e.g., 91%, 92%, 93%, 94%, 95%, 96%, 97/s, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3 or 10, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 90% or more (e.g., 91%, 92%, 93%, 94%, 95%, 96%, 97%, 98%, 99% or more) identity to SEQ ID NO: 19 and a light chain variable region comprising the amino acid sequence having 90% or more (e.g., 91%, 92%, 93%, 94%, 95%, 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, 21, 23, 25, 27, 29, 31 or 33; SEQ ID NO: 13; and SEQ ID NO: 14, respectively; and light chain CDR1, CDR2, and CDR3 sequences as set forth in SEQ ID NO: 15, SEQ ID NO: 16 or 17, and SEQ ID NO: 20, respectively) may be maintained.

In some embodiments, the antibody of the invention, or the antigen-binding fragment thereof, comprises (i) a heavy chain variable region comprising an amino acid sequence having 95% or more (e.g., 96%, 97%, 98%, 99% or more) identity to SEQ ID NO: 8 and a light chain variable region comprising the amino acid sequence having 95% or more (e.g., 96%, 97%, 98%, 99% or more) identity to SEQ ID NO: 9. Thereby, the CDR sequences as defined above (heavy chain CDR1, CDR2, and CDR3 sequences as set forth in SEQ ID NO: 1, SEQ ID NO: 2, and SEQ ID NO: 3 or 10, respectively; and light chain CDR1, CDR2, and CDR3 sequences as set forth in SEQ ID NO: 4, SEQ ID NO: 5 or 6, and SEQ ID NO: 7, respectively) may be maintained.

In some embodiments, the antibody of the invention, or the antigen-binding fragment thereof, comprises (i) a heavy chain variable region comprising an amino acid sequence having 95% or more (e.g., 96%, 97%, 98%, 99% or more) identity to SEQ ID NO: 19 and a light chain variable region comprising the amino acid sequence having 95% or more (e.g., 96%, 97%, 98%, 99% or more) identity to SEQ ID NO: 20. Thereby, the CDR sequences as defined above (heavy chain CDR1, CDR2, and CDR3 sequences as set forth in SEQ ID NO: 12, 21, 23, 25, 27, 29, 31 or 33; SEQ ID NO: 13; and SEQ ID NO: 14, respectively; and light chain CDR1, CDR2, and CDR3 sequences as set forth in SEQ ID NO: 15, SEQ ID NO: 16 or 17, and SEQ ID NO: 20, respectively) may be maintained.

More specifically, the antibody, or an antigen-binding fragment thereof, may comprise a heavy chain variable region comprising an amino acid sequence as set forth in SEQ ID NO: 8 and a light chain variable region comprising an amino acid sequence as set forth in SEQ ID NO: 9.

Alternatively, the antibody, or an antigen-binding fragment thereof, may comprise a heavy chain variable region comprising an amino acid sequence as set forth in SEQ ID NO: 11 and a light chain variable region comprising an amino acid sequence as set forth in SEQ ID NO: 9.

Alternatively, the antibody, or an antigen-binding fragment thereof, may comprise a heavy chain variable region comprising an amino acid sequence as set forth in SEQ ID NO: 19 and a light chain variable region comprising an amino acid sequence as set forth in SEQ ID NO: 20.

Alternatively, the antibody, or an antigen-binding fragment thereof, may comprise a heavy chain variable region comprising an amino acid sequence as set forth in SEQ ID NO: 22 and a light chain variable region comprising an amino acid sequence as set forth in SEQ ID NO: 20.

Alternatively, the antibody, or an antigen-binding fragment thereof, may comprise a heavy chain variable region comprising an amino acid sequence as set forth in SEQ ID NO: 24 and a light chain variable region comprising an amino acid sequence as set forth in SEQ ID NO: 20.

Alternatively, the antibody, or an antigen-binding fragment thereof, may comprise a heavy chain variable region comprising an amino acid sequence as set forth in SEQ ID NO: 26 and a light chain variable region comprising an amino acid sequence as set forth in SEQ ID NO: 20.

Alternatively, the antibody, or an antigen-binding fragment thereof, may comprise a heavy chain variable region comprising an amino acid sequence as set forth in SEQ ID NO: 28 and a light chain variable region comprising an amino acid sequence as set forth in SEQ ID NO: 20.

Alternatively, the antibody, or an antigen-binding fragment thereof, may comprise a heavy chain variable region comprising an amino acid sequence as set forth in SEQ ID NO: 30 and a light chain variable region comprising an amino acid sequence as set forth in SEQ ID NO: 20.

Alternatively, the antibody, or an antigen-binding fragment thereof, may comprise a heavy chain variable region comprising an amino acid sequence as set forth in SEQ ID NO: 32 and a light chain variable region comprising an amino acid sequence as set forth in SEQ ID NO: 20.

Alternatively, the antibody, or an antigen-binding fragment thereof, may comprise a heavy chain variable region comprising an amino acid sequence as set forth in SEQ ID NO: 34 and a light chain variable region comprising an amino acid sequence as set forth in SEQ ID NO: 20.

The CDR and VHNL sequences of exemplified antibodies of the invention, namely antibodies MPF5_VH117D (MPF5) and MPF5_VH117H, MPE33, MPE33_S36A, MPE33_N34Q, MPE33_N34S, MPE33_C38S, MPE33_C38A, MPE33_C38Y and MPE33_N34S_C38Y are shown in Table 1 below.

TABLE 1
CDR and VH/VL sequences (SEQ ID NOs) of exemplified antibodies of the invention.

| | Heavy chain | | | | Light chain | | | |
|---|---|---|---|---|---|---|---|---|
| Antibody name | CDR1 | CDR2 | CDR3 | VH | CDR1 | CDR2 | CDR3 | VL |
| MPF5_VH117D (MPF5) | 1 | 2 | 3 | 8 | 4 | 5/6 | 7 | 9 |
| MPF5_VH117H | 1 | 2 | 10 | 10 | 4 | 5/6 | 7 | 9 |
| MPE33 | 12 | 13 | 14 | 19 | 15 | 16/17 | 18 | 20 |
| MPE33_S36A | 21 | 13 | 14 | 22 | 15 | 16/17 | 18 | 20 |
| MPE33_N34Q | 23 | 13 | 14 | 24 | 15 | 16/17 | 18 | 20 |
| MPE33_N34S | 25 | 13 | 14 | 26 | 15 | 16/17 | 18 | 20 |
| MPE33_C38S | 27 | 13 | 14 | 28 | 15 | 16/17 | 18 | 20 |
| MPE33_C38A | 29 | 13 | 14 | 30 | 15 | 16/17 | 18 | 20 |
| MPE33_C38Y | 31 | 13 | 14 | 32 | 15 | 16/17 | 18 | 20 |
| MPE33_N34S_C38Y | 33 | 13 | 14 | 34 | 15 | 16/17 | 18 | 20 |

In some embodiments, the antibody of the invention is a human antibody. In some embodiments, the antibody of the invention is a monoclonal antibody. For example, the antibody of the invention may be a human monoclonal antibody.

Antibodies of the invention can be of any isotype (e.g., IgA, IgG, IgM i.e. an α, γ or μ heavy chain). For example, the antibody may be of the IgG type. Within the IgG isotype, antibodies may be IgG1, IgG2, IgG3 or IgG4 subclass, for example IgG1. Antibodies of the invention may have a κ or a λ light chain. In some embodiments, the antibody is of IgG1 type and has a lambda or kappa light chain.

In some embodiments, the antibody is of the human IgG1 type. The antibody may be of any allotype. The term “allotype” refers to the allelic variation found among the IgG subclasses. For example, the antibody may be of the G1 m1 (or G1m(a)) allotype, of the G1m2 (or G1m(x)) allotype, of the G1m3 (or G1m(f)) allotype, and/or of the G1 m17 (or Gm(z)) allotype. The G1m3 and G1m17 allotypes are located at the same position in the CH1 domain (position 214 according to EU numbering). G1m3 corresponds to R214 (EU), while G1m17 corresponds to K214 (EU). The G1m1 allotype is located in the CH3 domain (at positions 356 and 358 (EU)) and refers to the replacements E356D and M358L. The G1m2 allotype refers to a replacement of the alanine in position 431 (EU) by a glycine. The G1 m1 allotype may be combined, for example, with the G1m3 or the G1m17 allotype. In some embodiments, the antibody is of the allotype G1m3 with no G1m1 (G1m3,−1). In some embodiments, the antibody is of the G1m17,1 allotype. In some embodiments, the antibody is of the G1m3,1 allotype. In some embodiments, the antibody is of the allotype G1m17 with no G1m1 (G1m17,−1). Optionally, these allotypes may be combined (or not combined) with the G1m2, G1m27 or G1m28 allotype. For example, the antibody may be of the G1m17,1,2 allotype.

In some embodiments, the antibody according to the present invention, or an antigen binding fragment thereof, comprises an Fc moiety. The Fc moiety may be derived from human origin, e.g. from human IgG1, IgG2, IgG3, and/or IgG4, such as human IgG1.

As used herein, the term “Fc moiety” refers to a sequence derived from the portion of an immunoglobulin heavy chain beginning in the hinge region just upstream of the papain cleavage site (e.g., residue 216 in native IgG, taking the first residue of heavy chain constant region to be 114) and ending at the C-terminus of the immunoglobulin heavy chain. Accordingly, an Fc moiety may be a complete Fc moiety or a portion (e.g., a domain) thereof. A complete Fc moiety comprises at least a hinge domain, a CH2 domain, and a CH3 domain (e.g., EU amino acid positions 216-446). An additional lysine residue (K) is sometimes present at the extreme C-terminus of the Fc moiety, but is often cleaved from a mature antibody.

Each of the amino acid positions within an Fc moiety have been numbered herein according to the art-recognized EU numbering system of Kabat, see e.g., by Kabat et al., in “Sequences of Proteins of Immunological Interest”, U.S. Dept. Health and Human Services, 1983 and 1987. The EU index or EU index as in Kabat or EU numbering refers to the numbering of the EU antibody (Edelman G M, Cunningham B A, Gall W E, Gottlieb P D, Rutishauser U, Waxdal M J. The covalent structure of an entire gammaG immunoglobulin molecule. Proc Natl Acad Sci U S A. 1969;63(1):78-85; Kabat E. A., National Institutes of Health (U.S.) Office of the Director, “Sequences of Proteins of Immunological Interest”, 5^{th} edition, Bethesda, MD : U.S. Dept. of Health and Human Services, Public Health Service, National Institutes of Health, 1991, hereby entirely incorporated by reference).

In some embodiments, in the context of the present invention an Fc moiety comprises at least one of: a hinge (e.g., upper, middle, and/or lower hinge region) domain, a CH2 domain, a CH3 domain, or a variant, portion, or fragment thereof. An Fc moiety may comprise at least a hinge domain, a CH2 domain or a CH3 domain. The Fc moiety may be a complete Fc moiety. The Fc moiety may also comprises one or more amino acid insertions, deletions, or substitutions relative to a naturally-occurring Fc moiety. For example, at least one of a hinge domain, CH2 domain or CH3 domain (or portion thereof) may be deleted. For example, an Fc moiety may comprise or consist of: (i) hinge domain (or portion thereof) fused to a CH2 domain (or portion thereof), (ii) a hinge domain (or portion thereof) fused to a CH3 domain (or portion thereof), (iii) a CH2 domain (or portion thereof) fused to a CH3 domain (or portion thereof), (iv) a hinge domain (or portion thereof), (v) a CH2 domain (or portion thereof), or (vi) a CH3 domain or portion thereof.

It will be understood by one of ordinary skill in the art that the Fc moiety may be modified such that it varies in amino acid sequence from the complete Fc moiety of a naturally occurring immunoglobulin molecule, while retaining at least one desirable function conferred by the naturally-occurring Fc moiety. Such functions include Fc receptor (FcR) binding, antibody half-life modulation, ADCC function, protein A binding, protein G binding, and complement binding. The portions of naturally occurring Fc moieties, which are responsible and/or essential for such functions are well known by those skilled in the art.

For example, to activate the complement cascade C1q binds to at least two molecules of IgG1 or one molecule of IgM, attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on hematopoietic cells. Fc receptors belong to the immunoglobulin superfamily, and were shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcεR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Therefore, the Fc moiety may provide cross-linking of receptors (FcγR). In humans, three classes of FcγR have been characterized, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is known to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologuous; and (iii) FcγRIII (CD16), which binds IgG with medium to low affinity and exists as two types: FcγRIIIA found on NK cells, macrophages, eosinophils and some monocytes and T cells and mediating ADCC and FcγRIIIB, which is highly expressed on neutrophils. FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. Importantly, 75% of all FcγRIIB is found in the liver (Ganesan, L. P. et al., 2012: FcγRIIb on liver sinusoidal endothelium clears small immune complexes. Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

Accordingly, antibodies, and antigen binding fragments thereof, of the invention may be able to bind to FcγRIIb, for example antibodies comprising an Fc moiety for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcγRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). Accordingly, the antibodies, or antigen binding fragments thereof, of the invention may comprise an engineered Fc moiety with the mutations S267E and L328F, in particular as described by Chu, S. Y. et al., 2008: Inhibition of B cell receptor-mediated activation of primary human B cells by coengagement of CD19 and FcγRIlb with Fc-engineered antibodies. Molecular Immunology 45, 3926-3933.

On B-cells it seems to function to suppress further immunoglobulin production and isotype switching to say for example the IgE class. On macrophages, FcγRIIB acts to inhibit phagocytosis as mediated through FcγRIIA. On eosinophils and mast cells the b form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into IgG1 and IgG4, reduces binding to FcγRI by 10³-fold and eliminated the human monocyte response to antibody-sensitized red blood cells (Armour, K. L., et al. Eur. J. Immunol. 29 (1999) 2613-2624). Regarding FcγRII binding, reduced binding for FcγRIIA is found e.g. for IgG mutation of at least one of E233-G236, P238, D265, N297, A327, P329, D270, Q295, A327, R292 and K414. Regarding FcγRIII binding, reduced binding to FcγRIIIA is found e.g. for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above mentioned mutation sites and methods for measuring binding to FcγRI and FcγRIIA are described in Shields, R. L., et al., J. Biol. Chem. 276 (2001) 6591-6604. For example, a single (S239D or I332E), double (S239D/I332E), and triple mutations (S239D/I332E/A330L) improved the affinity against human FcγRIIIa. Furthermore, the addition of the mutation G236A to S239D/I332E improved not only FcγRIIa:FcγRIIb ratio, but also enhanced binding to FcγRIIIa. Accordingly, the mutations G236A/S239D/A330L/I332E were described to enhance engagement of FcγRIIa and FcγRIIIa.

Regarding binding to the crucial FcγRII, two regions of native IgG Fc appear to be critical for interactions of FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

For example, the Fc moiety may comprise or consist of at least the portion of an Fc moiety that is known in the art to be required for FcRn binding or extended half-life. Alternatively or additionally, the Fc moiety of the antibody of the invention comprises at least the portion of known in the art to be required for Protein A binding and/or the Fc moiety of the antibody of the invention comprises at least the portion of an Fc molecule known in the art to be required for protein G binding. The Fc moiety may comprise at least the portion known in the art to be required for FcγR binding. As outlined above, an Fc moiety may thus at least comprise (i) the lower hinge site of native IgG Fc, in particular amino acid residues L, L, G, G (234-237, EU numbering), and (ii) the adjacent region of the CH2 domain of native IgG Fc, in particular a loop and strands in the upper CH2 domain adjacent to the lower hinge region, e.g. in a region of P331, for example a region of at least 3, 4, 5, 6, 7, 8, 9, or 10 consecutive amino acids in the upper CH2 domain of native IgG Fc around P331, e.g. between amino acids 320 and 340 (EU numbering) of native IgG Fc.

In some embodiments, the antibody, or antigen binding fragment thereof, according to the present invention comprises an Fc region. As used herein, the term “Fc region” refers to the portion of an immunoglobulin formed by two or more Fc moieties of antibody heavy chains. For example, the Fc region may be monomeric or “single-chain” Fc region (i.e., a scFc region). Single chain Fc regions are comprised of Fc moieties linked within a single polypeptide chain (e.g., encoded in a single contiguous nucleic acid sequence). Exemplary scFc regions are disclosed in WO 2008/143954 A2. The Fc region may be dimeric. A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties.

The Fc moieties of the Fc region may be of the same or different class and/or subclass. For example, the Fc moieties may be derived from an immunoglobulin (e.g., a human immunoglobulin) of an IgG1, IgG2, IgG3 or IgG4 subclass. The Fc moieties of the Fc region may be of the same class and subclass. However, the Fc region (or one or more Fc moieties of an Fc region) may also be chimeric, whereby a chimeric Fc region may comprise Fc moieties derived from different immunoglobulin classes and/or subclasses. For example, at least two of the Fc moieties of a dimeric or single-chain Fc region may be from different immunoglobulin classes and/or subclasses. Additionally or alternatively, the chimeric Fc regions may comprise one or more chimeric Fc moieties. For example, the chimeric Fc region or moiety may comprise one or more portions derived from an immunoglobulin of a first subclass (e.g., an IgG1, IgG2, or IgG3 subclass) while the remainder of the Fc region or moiety is of a different subclass. For example, an Fc region or moiety of an Fc polypeptide may comprise a CH2 and/or CH3 domain derived from an immunoglobulin of a first subclass (e.g., an IgG1, IgG2 or IgG4 subclass) and a hinge region from an immunoglobulin of a second subclass (e.g., an IgG3 subclass). For example, the Fc region or moiety may comprise a hinge and/or CH2 domain derived from an immunoglobulin of a first subclass (e.g., an IgG4 subclass) and a CH3 domain from an immunoglobulin of a second subclass (e.g., an IgG1, IgG2, or IgG3 subclass). For example, the chimeric Fc region may comprise an Fc moiety (e.g., a complete Fc moiety) from an immunoglobulin for a first subclass (e.g., an IgG4 subclass) and an Fc moiety from an immunoglobulin of a second subclass (e.g., an IgG1, IgG2 or IgG3 subclass). For example, the Fc region or moiety may comprise a CH2 domain from an IgG4 immunoglobulin and a CH3 domain from an IgG1 immunoglobulin. For example, the Fc region or moiety may comprise a CH1 domain and a CH2 domain from an IgG4 molecule and a CH3 domain from an IgG1 molecule. For example, the Fc region or moiety may comprise a portion of a CH2 domain from a particular subclass of antibody, e.g., EU positions 292-340 of a CH2 domain. For example, an Fc region or moiety may comprise amino acids a positions 292-340 of CH2 derived from an IgG4 moiety and the remainder of CH2 derived from an IgG1 moiety (alternatively, 292-340 of CH2 may be derived from an IgG1 moiety and the remainder of CH2 derived from an IgG4 moiety).

Moreover, an Fc region or moiety may (additionally or alternatively) for example comprise a chimeric hinge region. For example, the chimeric hinge may be derived, e.g. in part, from an IgG1, IgG2, or IgG4 molecule (e.g., an upper and lower middle hinge sequence) and, in part, from an IgG3 molecule (e.g., an middle hinge sequence). In another example, an Fc region or moiety may comprise a chimeric hinge derived, in part, from an IgG1 molecule and, in part, from an IgG4 molecule. In another example, the chimeric hinge may comprise upper and lower hinge domains from an IgG4 molecule and a middle hinge domain from an IgG1 molecule. Such a chimeric hinge may be made, for example, by introducing a proline substitution (Ser228Pro) at EU position 228 in the middle hinge domain of an IgG4 hinge region. In other embodiments, the chimeric hinge can comprise amino acids at EU positions 233-236 are from an IgG2 antibody and/or the Ser228Pro mutation, wherein the remaining amino acids of the hinge are from an IgG4 antibody (e.g., a chimeric hinge of the sequence ESKYGPPCPPCPAPPVAGP). Further chimeric hinges, which may be used in the Fc moiety of the antibody according to the present invention are described in US 2005/0163783 A1.

In some embodiments, the Fc moiety, or the Fc region, comprises or consists of an amino acid sequence derived from a human immunoglobulin sequence (e.g., from an Fc region or Fc moiety from a human IgG molecule). However, polypeptides may comprise one or more amino acids from another mammalian species. For example, a primate Fc moiety or a primate binding site may be included in the subject polypeptides. Alternatively, one or more murine amino acids may be present in the Fc moiety or in the Fc region.

In some embodiments, the antibody according to the present invention comprises, in particular in addition to an Fc moiety as described above, other parts derived from a constant region, in particular from a constant region of IgG, such as a constant region of (human) IgG1. The antibody according to the present invention may comprise, in particular in addition to an Fc moiety as described above, all other parts of the constant regions, in particular all other parts of the constant regions of IgG (such as (human) IgG1).

Example sequences of constant regions are the amino acid sequences according to SEQ ID NOs: 35-37. For example, the amino acid sequence of IgG1 CH1-CH2-CH3 is according to SEQ ID NO: 35 or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity.

As outlined above, an antibody according to the present invention may comprise a (complete) Fc region derived from human IgG1. In some embodiments, the antibody according to the present invention comprises, in particular in addition to a (complete) Fc region derived from human IgG1 also all other parts of the constant regions of IgG, such as all other parts of the constant regions of (human) IgG1.

In some embodiments, the antibody according to the present invention comprises a (complete) Fc moiety/Fc region, wherein the interaction/binding with FcR is not compromised. In general, binding of the antibody to an Fc receptor may be assessed by various methods known to the skilled person, such as ELISA (Hessell A J, Hangartner L, Hunter M, Havenith C E G, Beurskens F J, Bakker J M, Lanigan C M S, Landucci G, Forthal D N, Parren P W H I, et al.: Fc receptor but not complement binding is important in antibody protection against HIV. Nature 2007, 449:101-104; Grevys A, Bern M, Foss S, Bratlie D B, Moen A, Gunnarsen K S, Aase A, Michaelsen T E, Sandlie I, Andersen J T: Fc Engineering of Human IgG1 for Altered Binding to the Neonatal Fc Receptor Affects Fc Effector Functions. 2015, 194:5497-5508) or flow-cytometry (Perez L G, Costa M R, Todd C A, Haynes B F, Montefiori D C: Utilization of immunoglobulin G Fc receptors by human immunodeficiency virus type 1: a specific role for antibodies against the membrane-proximal external region of gp41. J Virol 2009, 83:7397-7410; Piccoli L, Campo I, Fregni C S, Rodriguez B M F, Minola A, Sallusto F, Luisetti M, Corti D, Lanzavecchia A: Neutralization and clearance of GM-CSF by autoantibodies in pulmonary alveolar proteinosis. Nat Commun 2015, 6:1-9).

In general, the antibody according to the present invention may be glycosylated. N-linked glycans attached to the CH2 domain of a heavy chain, for instance, can influence C1q and FcR binding, with glycosylated antibodies having lower affinity for these receptors. Accordingly, the CH2 domain of the Fc moiety of the antibody according to the present invention may comprise one or more mutations, in which a glycosylated residue is substituted by a non-glycosylated residue. For example, the antibody's glycans do not lead to a human immunogenic response after administration.

Furthermore, the antibody according to the present invention can be modified by introducing (random) amino acid mutations into particular region of the CH2 or CH3 domain of the heavy chain in order to alter their binding affinity for FcR and/or their serum half-life in comparison to unmodified antibodies. Examples of such modifications include, but are not limited to, substitutions of at least one amino acid from the heavy chain constant region selected from the group consisting of amino acid residues 250, 314, and 428. Further examples of such Fc modifications are described in Saxena A, Wu D. Advances in Therapeutic Fc Engineering -Modulation of IgG-Associated Effector Functions and Serum Half-life. Front Immunol. 2016;7:580, which is incorporated herein by reference. In some embodiments, the antibody may comprise the “YTE” mutations (M252Y/S254T/T256E; EU numbering). In some embodiments, the antibody may comprise the mutations M428L and/or N434S in the heavy chain constant region (EU numbering).

Antibodies of the invention also include hybrid antibody molecules that comprise the six CDRs from an antibody of the invention as defined above and one or more CDRs from another antibody to an antigen. For example, the antibody may be bispecific. A bispecific (or multispecific) antibody, or antigen-binding fragment thereof, according to the present invention may comprise at least one specificity (antigen-binding site of an antibody) as described herein. In some embodiments, the bispecific (or multispecific) antibody, or antigen-binding fragment thereof, binds to two distinct epitopes of MPV F protein. To this end, the specificities of two distinct antibodies of the present invention may be combined, e.g. as described below for the combination of antibodies (but instead of a “cocktail” a bispecific—or multispecific—antibody may be provided with the combined specificities).

Variant antibodies are also included within the scope of the invention. Thus, variants of the sequences recited in the application are also included within the scope of the invention. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Antibodies of the invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies of the invention may be immunogenic in non-human (or heterologous) hosts e.g., in mice. In particular, the antibodies may have an idiotope that is immunogenic in non-human hosts, but not in a human host. In particular, antibodies of the invention for human use include those that cannot be easily isolated from hosts such as mice, goats, rabbits, rats, non-primate mammals, etc. and cannot generally be obtained by humanization or from xeno-mice.

### Nucleic Acids

In another aspect, the invention also provides a nucleic acid molecule comprising a polynucleotide encoding the antibody according to the present invention, or an antigen-binding fragment thereof, as described above.

In some embodiments, the nucleic acid molecule comprises one or more polynucleotide(s) encoding the exemplified antibodies of the invention (e.g., as described in Table 1 above), or a sequence variant thereof as described herein (e.g., having at least 70%, 71° /o, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity as described above).

Exemplified nucleic acid sequences encoding the CDR and VHNL sequences of exemplified antibodies of the invention are shown in Table 2 below.

TABLE 2
Exemplified nucleic acid CDR and VH/VL sequences (SEQ
ID NOs) of exemplified antibodies of the invention.

| Antibody | Heavy chain | | | | Light chain | | | |
|---|---|---|---|---|---|---|---|---|
| name | CDR1 | CDR2 | CDR3 | VH | CDR1 | CDR2 | CDR3 | VL |
| MPF5 | 38 | 39 | 40 | 45 | 41 | 42/43 | 44 | 46 |
| MPE33 | 47 | 48 | 49 | 54 | 50 | 51/52 | 53 | 55 |

For example, the nucleic acid molecule may comprise:
(i) a polynucleotide according to any one of SEQ ID NOs 45 or 54; and a polynucleotide according to any one of SEQ ID NOs 46 or 55; or
(ii) a polynucleotide according to any one of SEQ ID NOs 38 or 47; a polynucleotide according to any one of SEQ ID NOs 39 or 48; a polynucleotide according to any one of SEQ ID NOs 40 or 49; a polynucleotide according to any one of SEQ ID NOs 41 or a polynucleotide according to any one of SEQ ID NOs 42, 43, 51 or 52; and a polynucleotide according to any one of SEQ ID NOs 44 or 53.

Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA. Nucleic acids may encode the light chain and/or the heavy chain of an antibody. In other words, the light chain and the heavy chain of the antibody may be encoded by the same nucleic acid molecule (e.g., in bicistronic manner). Alternatively, the light chain and the heavy chain of the antibody may be encoded by distinct nucleic acid molecules.

Due to the redundancy of the genetic code, the present invention also comprises sequence variants of nucleic acid sequences, which encode the same amino acid sequences. The polynucleotide encoding the antibody (or the complete nucleic acid molecule) may be optimized for expression of the antibody. For example, codon optimization of the nucleotide sequence may be used to improve the efficiency of translation in expression systems for the production of the antibody. Moreover, the nucleic acid molecule may comprise heterologous elements (i.e., elements, which in nature do not occur on the same nucleic acid molecule as the coding sequence for the (heavy or light chain of) an antibody. For example, a nucleic acid molecule may comprise a heterologous promotor, a heterologous enhancer, a heterologous UTR (e.g., for optimal translation/expression), a heterologous Poly-A-tail, and the like.

A nucleic acid molecule is a molecule comprising nucleic acid components. The term nucleic acid molecule usually refers to DNA or RNA molecules. It may be used synonymous with the term “polynucleotide”, i.e. the nucleic acid molecule may consist of a polynucleotide encoding the antibody. Alternatively, the nucleic acid molecule may also comprise further elements in addition to the polynucleotide encoding the antibody. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g., labels) or can introduce tags (e.g., for purification purposes). Alternatively, a mutation in a nucleic acid sequence may be “silent”, i.e. not reflected in the amino acid sequence due to the redundancy of the genetic code. In general, mutations can be introduced in specific sites or can be introduced at random, followed by selection (e.g., molecular evolution). For instance, one or more nucleic acids encoding any of the light or heavy chains of an (exemplary) antibody can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the polynucleotide encoding the antibody, or an antigen-binding fragment thereof, (or the (complete) nucleic acid molecule) may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, JCat: a novel tool to adapt codon usage of a target gene to its potential expression host. Nucleic Acids Res. 2005 Jul 1;33(Web Server issue):W526-31; or, for example, Genscript's OptimumGene™ algorithm (as described in US 2011/0081708 A1).

For example, the nucleic acid molecule of the invention may comprise a nucleic acid sequence as set forth in any one of SEQ ID NOs 38-55; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

The present invention also provides a combination of a first and a second nucleic acid molecule, wherein the first nucleic acid molecule comprises a polynucleotide encoding the heavy chain of the antibody, or an antigen-binding fragment thereof, of the present invention; and the second nucleic acid molecule comprises a polynucleotide encoding the corresponding light chain of the same antibody, or the same antigen-binding fragment thereof. The above description regarding the (general) features of the nucleic acid molecule of the invention applies accordingly to the first and second nucleic acid molecule of the combination. Accordingly, one or both of the polynucleotides encoding the heavy and/or light chain(s) of the antibody, or an antigen-binding fragment thereof, may be codon-optimized. For example, the combination may comprise a nucleic acid sequence as set forth in any one of SEQ ID NOs 38 — 55; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

The present invention also provides a combination of a first and a second nucleic acid molecule, wherein
the first nucleic acid molecule comprises a polynucleotide encoding the heavy chain of an antibody, or an antigen-binding fragment thereof, the polynucleotide comprising: (a) nucleotide sequences according to SEQ ID NOs 38, 39 and 40; or (b) nucleotide sequences according to SEQ ID NOs 47, 48 and 49; and
the second nucleic acid molecule comprises a polynucleotide encoding the light chain of an antibody, or an antigen-binding fragment thereof, the polynucleotide comprising: (c) nucleotide sequences according to SEQ ID NOs 41, 42 (or 43) and 44; or (d) nucleotide sequences according to SEQ ID NOs 50, 51 (or 52) and 53.

Such a combination usually encodes the antibody, or an antigen-binding fragment thereof, of the present invention as described above. Again, the above description regarding the (general) features of the nucleic acid molecule of the invention applies accordingly to the first and second nucleic acid molecule of the combination.

### Vector

Further included within the scope of the invention are vectors, for example, expression vectors, comprising a nucleic acid molecule according to the present invention. Usually, a vector comprises a nucleic acid molecule as described above.

The present invention also provides a combination of a first and a second vector, wherein the first vector comprises a first nucleic acid molecule as described above (for the combination of nucleic acid molecules) and the second vector comprises a second nucleic acid molecule as described above (for the combination of nucleic acid molecules).

A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multi cloning site, a heterologous promotor, a heterologous enhancer, a heterologous selection marker (to identify cells comprising said vector in comparison to cells not comprising said vector) and the like. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a (heavy and/or light chain of a) desired antibody according to the present invention. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

### Cells

In a further aspect, the present invention also provides cell expressing the antibody according to the present invention, or an antigen-binding fragment thereof; and/or comprising the vector (or the combination of vectors) according the present invention.

Examples of such cells include but are not limited to, eukaryotic cells, e.g., yeast cells, animal cells or plant cells. Other examples of such cells include but are not limited, to prokaryotic cells, e.g. E. coli. In some embodiments, the cells are mammalian cells, such as a mammalian cell line. Examples include human cells, CHO cells, HEK293T cells, PER.C6 cells, NS0 cells, human liver cells, myeloma cells or hybridoma cells.

The cell may be transfected with a vector according to the present invention, for example with an expression vector. The term “transfection” refers to the introduction of nucleic acid molecules, such as DNA or RNA (e.g. mRNA) molecules, into cells, e.g. into eukaryotic or prokaryotic cells. In the context of the present invention, the term “transfection” encompasses any method known to the skilled person for introducing nucleic acid molecules into cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral.

Moreover, the cells of the present invention may be transfected stably or transiently with the vector according to the present invention, e.g. for expressing the antibody according to the present invention. In some embodiments, the cells are stably transfected with the vector according to the present invention encoding the antibody according to the present invention. In other embodiments, the cells are transiently transfected with the vector according to the present invention encoding the antibody according to the present invention.

Accordingly, the present invention also provides a recombinant host cell, which heterologously expresses the antibody of the invention or the antigen-binding fragment thereof. For example, the cell may be of another species than the antibody (e.g., CHO cells expressing human antibodies). In some embodiments, the cell type of the cell does not express (such) antibodies in nature. Moreover, the host cell may impart a post-translational modification (PTM; e.g., glycosylation) on the antibody that is not present in their native state. Such a PTM may result in a functional difference (e.g., reduced immunogenicity). Accordingly, the antibody of the invention, or the antigen-binding fragment thereof, may have a post-translational modification, which is distinct from the naturally produced antibody (e.g., an antibody of an immune response in a human).

### Production of Antibodies

Antibodies according to the invention can be made by any method known in the art. For example, the general methodology for making monoclonal antibodies using hybridoma technology is well known (Kohler, G. and Milstein, C., 1975; Kozbar et al. 1983). In some embodiments, the alternative EBV immortalization method described in WO2004/076677 is used.

In some embodiments, the method as described in WO 2004/076677, which is incorporated herein by reference, is used. In this method B cells producing the antibody of the invention are transformed with EBV and a polyclonal B cell activator. Additional stimulants of cellular growth and differentiation may optionally be added during the transformation step to further enhance the efficiency. These stimulants may be cytokines such as IL-2 and IL-15. In one aspect, IL-2 is added during the immortalization step to further improve the efficiency of immortalization, but its use is not essential. The immortalized B cells produced using these methods can then be cultured using methods known in the art and antibodies isolated therefrom.

Another exemplified method is described in WO 2010/046775. In this method plasma cells are cultured in limited numbers, or as single plasma cells in microwell culture plates. Antibodies can be isolated from the plasma cell cultures. Further, from the plasma cell cultures, RNA can be extracted and PCR can be performed using methods known in the art. The VH and VL regions of the antibodies can be amplified by RT-PCR (reverse transcriptase PCR), sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

Standard techniques of molecular biology may be used to prepare DNA sequences encoding the antibodies of the present invention. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention. Eukaryotic, e.g., mammalian, host cell expression systems may be used for production of antibody molecules, such as complete antibody molecules. Suitable mammalian host cells include, but are not limited to, CHO, HEK293T, PER.C6, NS0, myeloma or hybridoma cells. Also, prokaryotic, e.g. bacterial host cell expression systems may be used for the production of antibody molecules, such as complete antibody molecules. Suitable bacterial host cells include, but are not limited to, E. coli cells.

The present invention also provides a process for the production of an antibody molecule according to the present invention comprising culturing a (heterologous) host cell comprising a vector encoding a nucleic acid of the present invention under conditions suitable for expression of protein from DNA encoding the antibody molecule of the present invention, and isolating the antibody molecule.

For production of the antibody comprising both heavy and light chains, a cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Antibodies according to the invention may be produced by (i) expressing a nucleic acid sequence according to the invention in a host cell, e.g. by use of a vector according to the present invention, and (ii) isolating the expressed antibody product. Additionally, the method may include (iii) purifying the isolated antibody. Transformed B cells and cultured plasma cells may be screened for those producing antibodies of the desired specificity or function.

The screening step may be carried out by any immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

The immortalized B cell clones or the transfected host-cells of the invention can be used in various ways e.g., as a source of monoclonal antibodies, as a source of nucleic acid (DNA or mRNA) encoding a monoclonal antibody of interest, for research, etc.

The invention also provides a composition comprising immortalized B memory cells or transfected host cells that produce antibodies according to the present invention.

The immortalized B cell clone or the cultured plasma cells of the invention may also be used as a source of nucleic acid for the cloning of antibody genes for subsequent recombinant expression. Expression from recombinant sources may be more common for pharmaceutical purposes than expression from B cells or hybridomas e.g., for reasons of stability, reproducibility, culture ease, etc.

Thus the invention also provides a method for preparing a recombinant cell, comprising the steps of: (i) obtaining one or more nucleic acids (e.g., heavy and/or light chain mRNAs) from the B cell clone or the cultured plasma cells that encodes the antibody of interest; (ii) inserting the nucleic acid into an expression vector and (iii) transfecting the vector into a (heterologous) host cell in order to permit expression of the antibody of interest in that host cell.

Similarly, the invention also provides a method for preparing a recombinant cell, comprising the steps of: (i) sequencing nucleic acid(s) from the B cell clone or the cultured plasma cells that encodes the antibody of interest; and (ii) using the sequence information from step (i) to prepare nucleic acid(s) for insertion into a host cell in order to permit expression of the antibody of interest in that host cell. The nucleic acid may, but need not, be manipulated between steps (i) and (ii) to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest, wherein the nucleic acids are nucleic acids that were derived from an immortalized B cell clone or a cultured plasma cell of the invention. Thus the procedures for first preparing the nucleic acid(s) and then using it to transfect a host cell can be performed at different times by different people in different places (e.g., in different countries).

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

Methods for obtaining and sequencing immunoglobulin genes from B cells or plasma cells are well known in the art (e.g., see Chapter 4 of Kuby Immunology, 4th edition, 2000).

The transfected host cell may be a eukaryotic cell, including yeast and animal cells, particularly mammalian cells (e.g., CHO cells, NS0 cells, human cells such as PER.C6 or HKB-11 cells, myeloma cells, or a human liver cell), as well as plant cells. In some embodiments, the transfected host cell is a mammalian cell, such as a human cell. In some embodiments, expression hosts can glycosylate the antibody of the invention, particularly with carbohydrate structures that are not themselves immunogenic in humans. In some embodiments the transfected host cell may be able to grow in serum-free media. In further embodiments the transfected host cell may be able to grow in culture without the presence of animal-derived products. The transfected host cell may also be cultured to give a cell line.

The invention also provides a method for preparing one or more nucleic acid molecules (e.g., heavy and light chain genes) that encode an antibody of interest, comprising the steps of: (i) preparing an immortalized B cell clone or culturing plasma cells according to the invention; (ii) obtaining from the B cell clone or the cultured plasma cells nucleic acid that encodes the antibody of interest. Further, the invention provides a method for obtaining a nucleic acid sequence that encodes an antibody of interest, comprising the steps of: (i) preparing an immortalized B cell clone or culturing plasma cells according to the invention; (ii) sequencing nucleic acid from the B cell clone or the cultured plasma cells that encodes the antibody of interest.

The invention further provides a method of preparing nucleic acid molecule(s) that encode an antibody of interest, comprising the step of obtaining the nucleic acid that was obtained from a transformed B cell clone or cultured plasma cells of the invention. Thus the procedures for first obtaining the B cell clone or the cultured plasma cell, and then obtaining nucleic acid(s) from the B cell clone or the cultured plasma cells can be performed at different times by different people in different places (e.g., in different countries).

The invention also comprises a method for preparing an antibody (e.g., for pharmaceutical use) according to the present invention, comprising the steps of: (i) obtaining and/or sequencing one or more nucleic acids (e.g., heavy and light chain genes) from the selected B cell clone or the cultured plasma cells expressing the antibody of interest; (ii) inserting the nucleic acid(s) into or using the nucleic acid(s) sequence(s) to prepare an expression vector; (iii) transfecting a host cell that can express the antibody of interest; (iv) culturing or sub-culturing the transfected host cells under conditions where the antibody of interest is expressed; and, optionally, (v) purifying the antibody of interest.

The invention also provides a method of preparing the antibody of interest comprising the steps of: culturing or sub-culturing a transfected host cell population, e.g. a stably transfected host cell population, under conditions where the antibody of interest is expressed and, optionally, purifying the antibody of interest, wherein said transfected host cell population has been prepared by (i) providing nucleic acid(s) encoding a selected antibody of interest that is produced by a B cell clone or cultured plasma cells prepared as described above, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest. Thus the procedures for first preparing the recombinant host cell and then culturing it to express antibody can be performed at very different times by different people in different places (e.g., in different countries).

### Pharmaceutical Composition

The present invention also provides a pharmaceutical composition comprising one or more of:
(i) the antibody of the present invention, or an antigen-binding fragment thereof;
(ii) the nucleic acid or the combination of nucleic acids of the present invention;
(iii) the vector or the combination of vectors of the present invention; and/or
(iv) the cell expressing the antibody according to the present invention or comprising the vector according to the present invention
   and, optionally, a pharmaceutically acceptable excipient, diluent or carrier.

In other words, the present invention also provides a pharmaceutical composition comprising the antibody according to the present invention, the nucleic acid according to the present invention, the vector according to the present invention and/or the cell according to the present invention.

The pharmaceutical composition may optionally also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In some embodiments, the pharmaceutically acceptable carrier, diluent and/or excipient in the pharmaceutical composition according to the present invention is not an active component in respect to MPV infection.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin®, for reconstitution with sterile water containing a preservative). The composition may be prepared for topical administration e.g., as an ointment, cream or powder. The composition may be prepared for oral administration e.g., as a tablet or capsule, as a spray, or as a syrup (optionally flavored). The composition may be prepared for pulmonary administration e.g., as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a subject. For example, a lyophilized antibody may be provided in kit form with sterile water or a sterile buffer.

In some embodiments, the (only) active ingredient in the composition is the antibody according to the present invention. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions of the invention generally have a pH between 5.5 and 8.5, in some embodiments this may be between 6 and 8, for example about 7. The pH may be maintained by the use of a buffer. The composition may be sterile and/or pyrogen free. The composition may be isotonic with respect to humans. In some embodiments pharmaceutical compositions of the invention are supplied in hermetically-sealed containers.

Within the scope of the invention are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody may be in dry form, for reconstitution before use with an appropriate sterile liquid.

A vehicle is typically understood to be a material that is suitable for storing, transporting, and/or administering a compound, such as a pharmaceutically active compound, in particular the antibodies according to the present invention. For example, the vehicle may be a physiologically acceptable liquid, which is suitable for storing, transporting, and/or administering a pharmaceutically active compound, in particular the antibodies according to the present invention. Once formulated, the compositions of the invention can be administered directly to the subject. In some embodiments the compositions are adapted for administration to mammalian, e.g., human subjects.

The pharmaceutical compositions of this invention may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions of the invention. Optionally, the pharmaceutical composition may be prepared for oral administration, e.g. as tablets, capsules and the like, for topical administration, or as injectable, e.g. as liquid solutions or suspensions. In some embodiments, the pharmaceutical composition is an injectable. Solid forms suitable for solution in, or suspension in, liquid vehicles prior to injection are also encompassed, for example the pharmaceutical composition may be in lyophilized form.

For injection, e.g. intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is an antibody, a peptide, a nucleic acid molecule, or another pharmaceutically useful compound according to the present invention that is to be given to an individual, administration is usually in an “effective amount”, e.g. in a “prophylactically effective amount” or a “therapeutically effective amount” (as the case may be), this being sufficient to show benefit to the individual. The actual amount administered, and rate and time-course of administration, will depend on the nature and severity of what is being treated. For injection, the pharmaceutical composition according to the present invention may be provided for example in a pre-filled syringe.

The inventive pharmaceutical composition as defined above may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the inventive transporter cargo conjugate molecule as defined above, is combined with emulsifying and suspending agents. If desired, certain sweetening, flavoring or coloring agents may also be added.

The inventive pharmaceutical composition may also be administered topically, especially when the target of treatment includes areas or organs readily accessible by topical application, e.g. including accessible epithelial tissue. Suitable topical formulations are readily prepared for each of these areas or organs. For topical applications, the inventive pharmaceutical composition may be formulated in a suitable ointment, containing the inventive pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the inventive pharmaceutical composition can be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule. In particular, the pharmaceutical composition may be provided as single-dose product. In some embodiments, the amount of the antibody in the pharmaceutical composition—in particular if provided as single-dose product—does not exceed 200 mg, for example it does not exceed 100 mg or 50 mg.

For a single dose, e.g. a daily, weekly or monthly dose, the amount of the antibody in the pharmaceutical composition according to the present invention, may not exceed 1 g or 500 mg. In some embodiments, for a single dose, the amount of the antibody in the pharmaceutical composition according to the present invention, may not exceed 200 mg, or 100 mg. For example, for a single dose, the amount of the antibody in the pharmaceutical composition according to the present invention, may not exceed 50 mg.

Pharmaceutical compositions typically include an “effective” amount of one or more antibodies of the invention, i.e. an amount that is sufficient to treat, ameliorate, attenuate, reduce or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician. For purposes of the present invention, an effective dose may generally be from about 0.005 to about 100 mg/kg, for example from about 0.0075 to about 50 mg/kg or from about 0.01 to about 10 mg/kg. In some embodiments, the effective dose will be from about 0.02 to about 5 mg/kg, of the antibody of the present invention (e.g. amount of the antibody in the pharmaceutical composition) in relation to the bodyweight (e.g., in kg) of the individual to which it is administered.

Moreover, the pharmaceutical composition according to the present invention may also comprise an additional active component, which may be a further antibody or a component, which is not an antibody. Accordingly, the pharmaceutical composition according to the present invention may comprise one or more of the additional active components.

The antibody according to the present invention can be present either in the same pharmaceutical composition as the additional active component or, alternatively, the antibody according to the present invention is comprised by a first pharmaceutical composition and the additional active component is comprised by a second pharmaceutical composition different from the first pharmaceutical composition. Accordingly, if more than one additional active component is envisaged, each additional active component and the antibody according to the present invention may be comprised in a different pharmaceutical composition. Such different pharmaceutical compositions may be administered either combined/simultaneously or at separate times or at separate locations (e.g. separate parts of the body).

The antibody according to the present invention and the additional active component may provide an additive therapeutic effect, such as a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

In other embodiments, the pharmaceutical composition according to the present invention may not comprise an additional active component (in addition to the antibody of the invention or respective nucleic acids, vectors or cells as described above).

In some embodiments, a composition of the invention may include antibodies of the invention, wherein the antibodies may make up at least 50% by weight (e.g., 60%, 70%, 75%, 80%, 85%, 90%, 95%, 96%, 97%, 98%, 99% or more) of the total protein in the composition. In the composition of the invention, the antibodies may be in purified form.

The present invention also provides a method of preparing a pharmaceutical composition comprising the steps of: (i) preparing an antibody of the invention; and (ii) admixing the purified antibody with one or more pharmaceutically acceptable excipients, diluents or carriers.

In other embodiments, a method of preparing a pharmaceutical composition comprises the step of: admixing an antibody with one or more pharmaceutically-acceptable carriers, wherein the antibody is a monoclonal antibody that was obtained from a transformed B cell or a cultured plasma cell of the invention.

As an alternative to delivering antibodies or B cells for therapeutic purposes, it is possible to deliver nucleic acid (typically DNA) that encodes the monoclonal antibody of interest derived from the B cell or the cultured plasma cells to a subject, such that the nucleic acid can be expressed in the subject in situ to provide a desired therapeutic effect. Suitable gene therapy and nucleic acid delivery vectors are known in the art.

Pharmaceutical compositions may include an antimicrobial, particularly if packaged in a multiple dose format. They may comprise detergent e.g., a Tween (polysorbate), such as Tween 80. Detergents are generally present at low levels e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

The compositions of the invention may also comprise one or more immunoregulatory agents. In some embodiments, one or more of the immunoregulatory agents include(s) an adjuvant.

### Combinations of Antibodies

In a further aspect, the present invention also provides a combination of distinct antibodies, or antigen-binding fragments thereof, binding to MPV F protein (and neutralizing MPV). In particular, the antibodies of such a combination may bind to distinct epitopes of MPV F protein. Whether antibodies bind to the same or distinct epitopes can be determined, for example, with competition studies known by the skilled person (and as described in the examples below).

In some embodiments, the combination of antibodies, or antigen-binding fragments thereof, comprises:
an antibody, or an antigen-binding fragment thereof, according to the present invention as described above; and
an antibody, or an antigen-binding fragment thereof, binding to (a distinct epitope of) MPV F protein.

Accordingly, the combination of antibodies, or antigen-binding fragments thereof, may comprise:
an antibody, or an antigen-binding fragment thereof, as described above, comprising (i) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 6, and SEQ ID NO: 7, respectively; and
an antibody, or an antigen-binding fragment thereof, binding to (a distinct epitope of) MPV F protein.

For example, the combination of antibodies, or antigen-binding fragments thereof, may comprise:
an antibody, or an antigen-binding fragment thereof, as described above, comprising the heavy chain CDR1, CDR2, and CDR3 sequences and the light chain CDR1, CDR2, and CDR3 of antibody MPF5 (MPF5_VH117D) or sequence variants thereof as described herein; and
an antibody, or an antigen-binding fragment thereof, binding to (a distinct epitope of) MPV F protein.

More specifically, the combination of antibodies, or antigen-binding fragments thereof, may comprise:
an antibody, or an antigen-binding fragment thereof, as described above, comprising the VH and VL sequences of antibody MPF5 (MPF5_VH117D) or sequence variants thereof as described herein; and
an antibody, or an antigen-binding fragment thereof, binding to (a distinct epitope of) MPV F protein.

Moreover, the combination of antibodies, or antigen-binding fragments thereof, may comprise:
an antibody, or an antigen-binding fragment thereof, as described above, comprising (i) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 16, and SEQ ID NO: 18, respectively; or (ii) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 17, and SEQ ID NO: 18, respectively; and
an antibody, or an antigen-binding fragment thereof, binding to (a distinct epitope of) MPV F protein.

For example, the combination of antibodies, or antigen-binding fragments thereof, may comprise:
an antibody, or an antigen-binding fragment thereof, as described above, comprising the heavy chain CDR1, CDR2, and CDR3 sequences and the light chain CDR1, CDR2, and CDR3 of antibody MPE33 or sequence variants thereof as described herein; and
an antibody, or an antigen-binding fragment thereof, binding to (a distinct epitope of) MPV F protein.

More specifically, the combination of antibodies, or antigen-binding fragments thereof, may comprise:
an antibody, or an antigen-binding fragment thereof, as described above, comprising the VH and VL sequences of antibody MPE33 or sequence variants thereof as described herein; and
an antibody, or an antigen-binding fragment thereof, binding to (a distinct epitope of) MPV F protein.

In particular, the combination of antibodies, or antigen-binding fragments thereof, may comprise two distinct antibodies, or antigen-binding fragments thereof, of the present invention as described herein. Such antibodies, or antigen-binding fragments thereof, of the present invention, may bind to distinct epitopes of MPV F-protein.

Accordingly, the combination of antibodies, or antigen-binding fragments thereof, may comprise:
an antibody, or an antigen-binding fragment thereof, as described above, comprising (i) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 6, and SEQ ID NO: 7, respectively; and
an antibody, or an antigen-binding fragment thereof, as described above, comprising (iii) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 16, and SEQ ID NO: 18, respectively; or (iv) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 17, and SEQ ID NO: 18, respectively.

For example, the combination of antibodies, or antigen-binding fragments thereof, may comprise:
an antibody, or an antigen-binding fragment thereof, as described above, comprising the heavy chain CDR1, CDR2, and CDR3 sequences and the light chain CDR1, CDR2, and CDR3 of antibody MPF5 (MPF5_VH117D) or a sequence variant thereof as described herein; and
an antibody, or an antigen-binding fragment thereof, as described above, comprising the heavy chain CDR1, CDR2, and CDR3 sequences and the light chain CDR1, CDR2, and CDR3 of antibody MPE33 or a sequence variant thereof as described herein.

More specifically, the combination of antibodies, or antigen-binding fragments thereof, may comprise:
an antibody, or an antigen-binding fragment thereof, as described above, comprising the VH and VL sequences of antibody MPF5 (MPF5_VH117D) or a sequence variant thereof as described herein; and
an antibody, or an antigen-binding fragment thereof, as described above, comprising the VH and VL sequences of antibody MPE33 or a sequence variant thereof as described herein.

For example, an antibody, or an antigen-binding fragment thereof, comprising the heavy chain CDR1 according to SEQ ID NO: 1, the heavy chain CDR2 according to SEQ ID NO: 2, and the heavy chain CDR3 according to SEQ ID NO: 3 or 10; and the light chain CDR1 according to SEQ ID NO: 4, the light chain CDR2 according to SEQ ID NO: 5 or 6, and the light chain CDR3 according to SEQ ID NO: 7; may be combined with an antibody, or an antigen-binding fragment thereof, comprising the heavy chain CDR1 according to any one of SEQ ID NOs 12, 21, 23, 25, 27, 29, 31 or 33, the heavy chain CDR2 according to SEQ ID NO: 13, and the heavy chain CDR3 according to SEQ ID NO: 14; and the light chain CDR1 according to SEQ ID NO: 15, the light chain CDR2 according to SEQ ID NO: 16 or 17, and the light chain CDR3 according to SEQ ID NO: 18.

More specifically, an antibody, or an antigen-binding fragment thereof, comprising the VH according to SEQ ID NO: 8 or 11 and the VL according to SEQ ID NO: 9, may be combined with an antibody, or an antigen-binding fragment thereof, comprising the VH according to SEQ ID NO: 19, 22, 24, 26, 28, 30, 32 or 34 and the VL according to SEQ ID NO: 20.

In the combination of antibodies, or antigen-binding fragments thereof, of the present invention the antibodies, or antigen-binding fragments thereof, may be provided in any form as described above, e.g. as proteins (antibodies), nucleic acids (encoding said antibodies), vectors (comprising said nucleic acids), cells (expressing said antibodies or comprising said vectors). Accordingly, the present invention provides a combination comprising
(i) two distinct antibodies of the present invention, or antigen-binding fragments thereof;
(ii) two distinct nucleic acids (or combinations of nucleic acids) of the present invention;
(iii) two distinct vectors (or combinations of vectors) of the present invention; or
(iv) two distinct cells expressing the two distinct antibodies according to the present invention or comprising the two distinct vectors according to the present invention.

It is understood that the combination of antibodies, or antigen-binding fragments thereof, of the present invention (in any form as described above, such as antibodies (proteins)) may be comprised in the same composition or distinct compositions, e.g. pharmaceutical composition(s) as described above. Accordingly, the present invention also provides a pharmaceutical composition comprising two distinct antibodies, or antigen-binding fragments of the present invention as described above.

### Medical Treatments and Other Uses

In a further aspect, the present invention provides the use of the antibody according to the present invention, or an antigen-binding fragment thereof, the nucleic acid molecule (or the combination of nucleic acid molecules) according to the present invention, the vector (or the combination of vectors) according to the present invention, the cell according to the present invention or the pharmaceutical composition according to the present invention as a medicament. In particular, the antibody according to the present invention, or an antigen-binding fragment thereof, the nucleic acid molecule (or the combination of nucleic acid molecules) according to the present invention, the vector (or the combination of vectors) according to the present invention, the cell according to the present invention, the pharmaceutical composition according to the present invention, or the combination of antibodies, or antigen-binding fragments thereof, of the present invention may be used in prophylaxis and/or treatment of MPV infection; or in (ii) diagnosis of MPV infection.

Accordingly, the present invention also provides a method of ameliorating or reducing MPV infection, or lowering the risk of MPV infection, comprising: administering to a subject in need thereof, a therapeutically effective amount of the antibody, or an antigen-binding fragment thereof, according to the present invention, the nucleic acid molecule (or the combination of nucleic acid molecules) according to the present invention, the vector (or the combination of vectors) according to the present invention, the cell according to the present invention or the pharmaceutical composition according to the present invention. Moreover, the present invention also provides the use of the antibody according to the present invention, or an antigen-binding fragment thereof, the nucleic acid molecule (or the combination of nucleic acid molecules) according to the present invention, the vector (or the combination of vectors) according to the present invention, the cell according to the present invention, the pharmaceutical composition according to the present invention, or the combination of antibodies, or antigen-binding fragments thereof, of the present invention in the manufacture of a medicament for prophylaxis, treatment or attenuation of MPV infection.

Prophylaxis of MPV infection refers in particular to prophylactic settings, wherein the subject was not diagnosed with MPV (either no diagnosis was performed or diagnosis results were negative) and/or the subject does not show symptoms of MPV infection. In therapeutic settings, in contrast, the subject is typically diagnosed with MPV infection and/or showing symptoms of MPV infection. Of note, the terms “treatment” and “therapy”/“therapeutic” of MPV infection include (complete) cure as well as attenuation/reduction of MPV infection and/or related symptoms.

In some embodiments the subject may be a human. One way of checking efficacy of therapeutic treatment involves monitoring disease symptoms after administration of the composition of the invention. Treatment can be a single dose schedule or a multiple dose schedule. In one embodiment, an antibody, antibody fragment, nucleic acid, vector, cell or composition according to the invention is administered to a subject in need of such treatment. Such a subject includes, but is not limited to, one who is particularly at risk of or susceptible to MPV infection, including, for example, an immunocompromised subject.

Antibodies and fragments thereof as described in the present invention may also be used for the diagnosis of MPV infection. Methods of diagnosis may include contacting an antibody with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood, such as plasma or serum. For example, the antibody, or an antigen-binding fragment thereof, may be contacted with an (isolated) blood sample (e.g., whole blood, plasma or serum). The methods of diagnosis may also include the detection of an antigen/antibody complex, in particular following the contacting of an antibody with a sample. Such a detection step is typically performed at the bench, i.e. without any contact to the human or animal body. Examples of detection methods are well-known to the person skilled in the art and include, e.g., ELISA (enzyme-linked immunosorbent assay). Accordingly, the diagnosis may be performed in vitro, for example by using an isolated sample as described above (and an in vitro detection step of an antigen/antibody complex). Accordingly, the antibody, or an antigen-binding fragment thereof, may be used in (in vitro) diagnosis of MPV infection.

Accordingly, the antibody of the present invention, or an antigen-binding fragment thereof, may be used in an (in vitro) method for detecting an MPV antigen. Likewise, the antibody of the present invention, or an antigen-binding fragment thereof, may be used in an (in vitro) method for binding MPV target protein/antigen, such as MPV F protein or antigenic fragments or variants thereof. Due to its specificity, the antibody of the present invention, or an antigen-binding fragment thereof, recognizes the MPV F protein, in particular in its pre-fusion conformation. For detecting an MPV antigen, the antibody may be brought in contact with a (isolated) sample (i.e., a sample to be tested for the presence of the antigen). By the specific binding of the antibody to its antigen (MPV F protein), an antibody/antigen complex is formed, which can be easily detected by methods known in the art.

Such a detection method may be used in the context of (in vitro) diagnosis (with samples isolated from a human or animal body), but also for testing other (e.g., production/manufacture) samples, such as vaccine samples. Accordingly, antibodies, antibody fragment, or variants thereof, as described in the present invention may also be used in a non-therapeutic/non-diagnostic context, e.g. in a vaccine development or manufacture. The present invention therefore also provides the use of the antibody of the present invention, or an antigen-binding fragment thereof, for testing vaccines, in particular whether the antigen (i.e., the desired antigen contained in the vaccine) is properly generated and/or folded (and/or in the correct conformation). Accordingly, the antibodies may be used for monitoring vaccine manufacture with the desired immunogenicity. To this end, the antibody may be brought in contact with the vaccine, e.g. as described above. Accordingly, the present invention also provides a method for testing anti-MPV vaccines, wherein the vaccine is contacted with the antibody, or an antigen-binding fragment thereof, and, optionally, the presence of antibody/antigen complexes is determined. Furthermore, the present invention also encompasses the use of the antibody of the present invention, or an antigen-binding fragment thereof, for monitoring the quality of anti-MPV vaccines by checking whether the vaccine contains the desired antigen, e.g. the F protein of MPV (for example in the pre-fusion conformation), or a fragment or variant thereof. More specifically, the antibody may be used to check the conformation of the antigen, or an epitope thereof, in a vaccine. As the antibodies of the present invention bind specifically to the pre-fusion F protein, detection of a significant amount of antibody/antigen complexes in a sample may imply that the sample contains MPV F protein primarily in the pre-fusion conformation. Furthermore, also modified versions of the antigen can be tested with the antibodies of the invention, such as fragments and variants of the MPV F protein, which may be useful in a vaccine.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows for Example 1 the binding properties of MPE33 and MPF5 antibodies to the hMPV F proteins in a pre-fusion and post-fusion conformation, in comparison to the reference antibody MPE8.

FIG. 2 shows for Example 2 the neutralization of four distinct hMPV strains A1/6621 (MPV A1), A2/VR8938 (MPV A2), B1/VR4702 (MPV B1), and B2/3817 (MPV B2) with antibodies MPE33 and MPF5 in comparison to the reference antibody MPE8v3.

FIG. 3 summarizes for Example 2 the EC50 required for neutralization of the four distinct hMPV strains A1/6621 (MPV A1), A2/VR8938 (MPV A2), B1/VR4702 (MPV B1), and B2/3817 (MPV B2) with antibodies MPE33 and MPF5 in comparison to the reference antibody MPE8v3.

FIG. 4 shows for Example 3 the binding affinities and EC50 values of antibodies MPF5_VH117D (MPF5), MPF5_VH117H, MPE33 and comparative antibody MPE8v3 for MPV pre-fusion F protein.

FIG. 5 shows the VH and VL (“VK”) sequences of antibodies MPF5_VH117D (MPF5) and MPF5_VH117H. The square indicates the position of the substitution at amino acid position VH117.

FIG. 6 shows for Example 4 the binding affinities and EC50 values of antibody MPE33 and its variants MPE33_S36A, MPE33_N34Q, MPE33_N34S, MPE33_C38S, MPE33_C38A, MPE33_C38Y and MPE33_N34S_C38Y for MPV pre-fusion F protein.

FIG. 7 shows the VH and VL (“VK”) sequences of antibody MPE33. The squares indicate the positions of the substitutions at amino acid position N34, S36 and C38.

FIG. 8 shows for Example 5 the binding of antibodies MPE33, MPE33_S36A, MPE33_N34Q, MPE33_N34S, MPE33_C38S, MPE33_C38A, MPE33_C38Y, MPE33_N34S_C38Y, MPF5_VH117D (MPF5), MPF5_VH117H and comparative antibody MPE8 to cell-associated F-antigen of hMPV strains MPV_NL/1/99_F0-TM (upper line) and HMPV_Yokohama/JPN(P8527)2016 (middle line). In the lower line, mock transfection (control) is shown.

FIG. 9 shows for Example 6 the results of a competition study of antibodies MPF5, MPE33 and MPE8 for binding to MPV F-protein. The data show competition between the same antibodies (MPF5 vs. MPF5; MPE33 vs. MPE33; MPE8 vs. MPE8), as expected. In addition, panels MPF5 vs. MPE8 and MPE8 vs. MPF5 demonstrate competition between MPF5 and MPE8. No competition, however, could be found between MPE33 and MPE8, or MPE33 and MPF5.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Identification and Characterization of Human Monoclonal Antibodies MPF5 and MPE33

Human monoclonal antibodies MPF5 (also referred to as “MPF5_VH117D”) and MPE33 against MPV were isolated (cf. Traggiai E. et al., 2004, Nat Med 10(8): 871-5) from human patients. The antibodies were characterized by determining the nucleotide and amino acid sequences of its variable regions (MPF5 VH: SEQ ID NO: 8, MPF5 VL: SEQ ID NO: 9; MPE33 VH: SEQ ID NO: 19, MPE33 VL: SEQ ID NO: 20) and the complementarity determining regions (CRDs) therein (MPF5: SEQ ID NOs 1-5 and 7, or 1-4, 6 and 7, respectively; MPE33: SEQ ID NOs 12-16 and 18, or 12-15, 17 and 18, respectively). The VH and VL genes of MPF5 and MPE33 were cloned into IgG1 expression vectors and recombinant antibodies were produced by transient transfection of 293 Freestyle cells (293F). Supernatants from transfected cells were collected and IgG were affinity purified by Protein A chromatography. Accordingly, MPF5 and MPE33 are IgG1-type fully human monoclonal antibodies with the CDR, VH and VL sequences as described herein.

In order to test the binding of MPF5 and MPE33 to hMPV F protein, an ELISA assay was performed essentially as described in WO 2016/103238 A1 for testing the binding affinity of the antibodies to hMPV F protein in pre- vs. post-fusion conformation. For comparison, prior art antibody MPE8 (Corti et at, 2013, Cross-neutralization of four paramyxoviruses by a human monoclonal antibody. Nature 501: 439-443) was also tested in this experiment.

Briefly, Maxisorp ELISA plates were coated overnight with both conformationally stabilized pre and post fusion F protein antigens (F protein from CAN97-83 MPV strain, 1 μg/ml, 25 μl/well in PBS pH7). After 3 washes with PBS/Tween 0.01% (PBST), test antibodies were added starting at 10 μg/ml, titrated down 3-fold by 11 points, and incubated 2 hrs at room temperature. Plates were then washed 4 times in PBST and dispensed with alkaline phosphatase-labeled goat anti-human IgG polyclonal antibody (SouthernBiotech, 2 μg/ml, 25 μl/well) and further incubated 1 hr at RT. Following 4 washes with PBST, plates were developed by adding 50 μl/well of AP substrate (pNPP, Sigma) in carbonate buffer and read at 405 nm after 45 min.

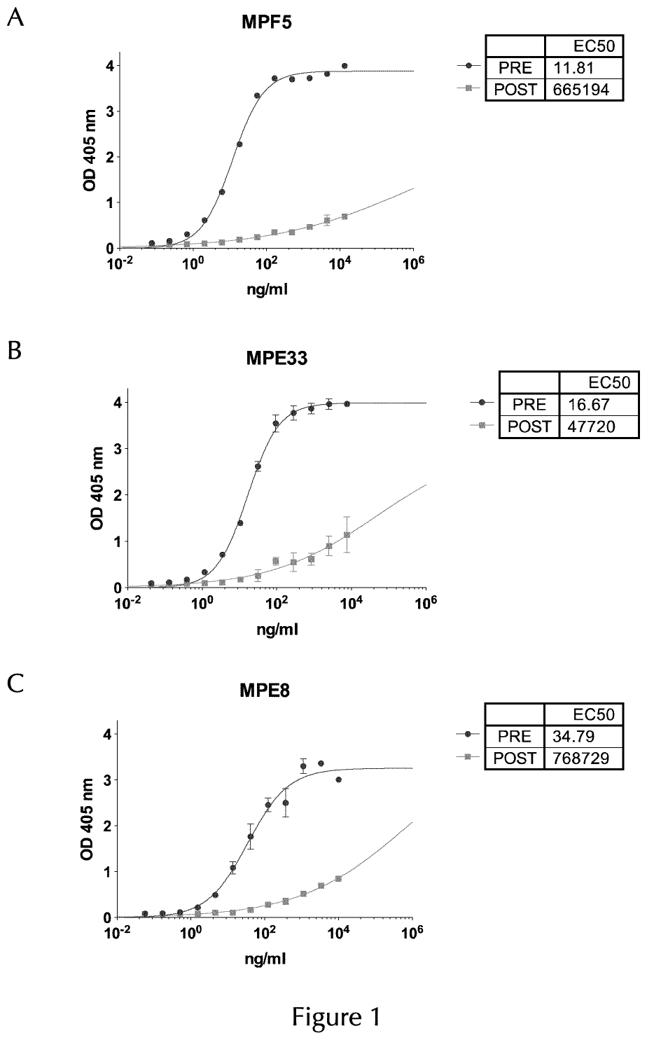

Results are shown in FIG. 1. All antibodies tested (MPF5, MPE33 and MPE8) showed high binding affinity to MPV pre-fusion F protein, but not to MPV post-fusion F protein. Accordingly, antibodies MPF5, MPE33 and MPE8 are specific for MPV pre-fusion F protein. Antibodies MPF5 and MPE33 of the present invention showed even lower EC50 values and, thus, higher binding affinity to MPV pre-fusion F protein as compared to prior art antibody MPE8.

### Example 2: MPF5 and MPE33 Effectively Neutralize Various Strains of MPV

Next, neutralization of various distinct strains of MPV was assessed with the antibodies MPF5 and MPE33 of the present invention as well with comparative antibody MPE8v3, which differs from control antibody MPE8 used in Example 1 in that it comprises a N113S mutation in the heavy chain variable region to remove a glycosylation site.

Briefly, culture supernatants with the antibodies were analyzed using a microneutralization assay based on infection of LLC-MK2 cells by hMPV strains A1/6621, A2/VR8938, B1/VR4702, and B2/3817. Neat supernatants were incubated with 0.239*10⁶ TCID50/ml (A1/6621), 0.0959*10⁶ TCID50/ml (A2/VR8938), 0.33*10⁶ TCID50/ml (B1/VR4702) and 0.0959*10⁶ TCID50/ml (B2/3817), respectively, of viruses for 1 h at room temperature before addition of LLC-MK2 target cells which were incubated for 14 days, respectively. Viable cells were detected using the WST-1 reagent (Roche). The EC50 was determined using the microneutralization assay described above with 100 TCID₅₀ of virus and viral infection was measured on day 6 or 7 by indirect immunofluorescence using an automated Pathway 855 analyser (BD). EC50 values were calculated by interpolation ,of neutralization curves fitted with a 4-parameter nonlinear regression with a variable slope.

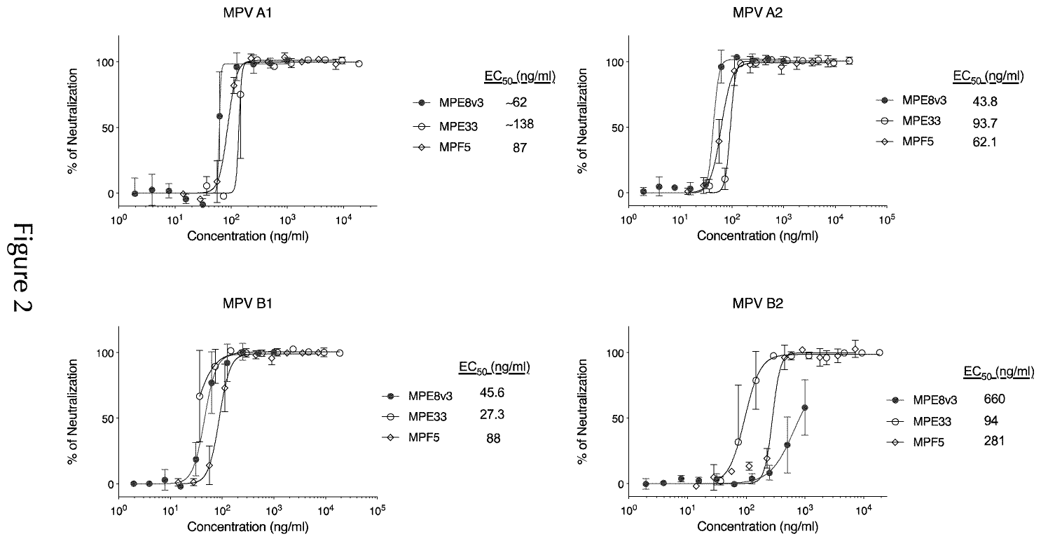

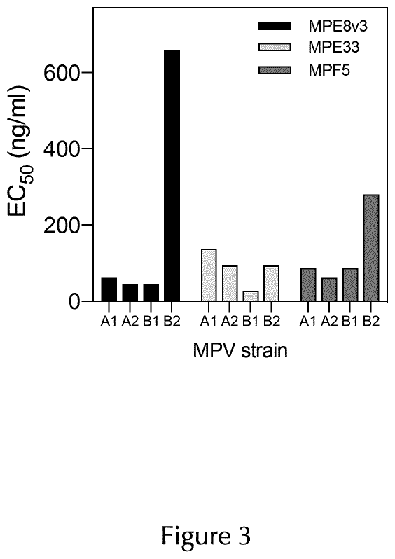

Results are shown in FIGS. 2 and 3. All antibodies effectively neutralized the four different MPV strains tested. Specifically, MPE33 and MPF5 showed a significantly better neutralization potency against MPV B2 strain, when compared to the reference mAb MPE8v3.

### Example 3: CDRH3 Mutant of MPF5 Shows Similarly High Binding Affinity to MPV Pre-Fusion F Protein

Next, variant antibody MPF5_VH117H of MPF5 was generated, which differs from MPF5 in that its CDRH3 sequence is according to SEQ ID NO: 10 and its VH sequence is according to SEQ ID NO: 11. The differences in the heavy chain sequence and CDRH3 of MPF5 are illustrated in FIG. 5.

The binding affinities of antibodies MPF5_VH117D (MPF5), MPF5_VH117H, MPE8v3 and MPE33 for MPV pre-fusion F protein were tested in an ELISA assay as described in Example 1.

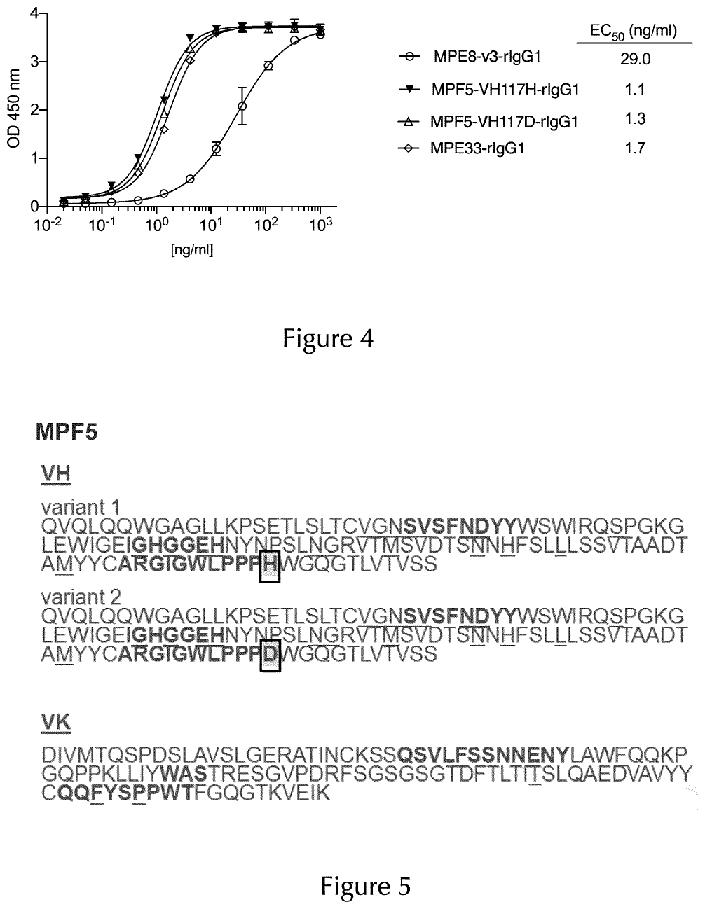

Results are shown in FIG. 4. While all tested antibodies specifically bound to MPV pre-fusion F protein, the binding affinities of the antibodies MPF5_VH117D (MPF5), MPF5_VH117H and MPE33 of the present invention were considerably higher than that of comparative antibody MPE8v3. Binding affinities of antibodies MPF5_VH117D (MPF5) and MPF5_VH117H were similarly high, indicating that the CDRH3 mutation did not impair binding affinity of MPF5 to MPV pre-fusion F protein.

### Example 4: CDRH1 Mutants of MPE33 Show Similarly High Binding Affinity to MPV Pre-Fusion F Protein

Next, the following variant antibodies of MPE33 were generated, which differ from MPE33 in the indicated CDRH1 sequence only:

TABLE 3

| | CDRH1 | VH |
|---|---|---|
| Antibody | (SEQ ID NO) | (SEQ ID NO) |
| MPE33 | 12 | 19 |
| MPE33_S36A | 21 | 22 |
| MPE33_N34Q | 23 | 24 |
| MPE33_N34S | 25 | 26 |
| MPE33_C38S | 27 | 28 |
| MPE33_C38A | 29 | 30 |
| MPE33_C38Y | 31 | 32 |
| MPE33_N34S_C38Y | 33 | 34 |

The positions of the mutated amino acids in the heavy chain sequence and CDRH3 of MPE33 are illustrated in FIG. 7.

The binding affinities of antibodies MPE33 and its variant antibodies described above for MPV pre-fusion F protein were tested in an ELISA assay as described in Example 1.

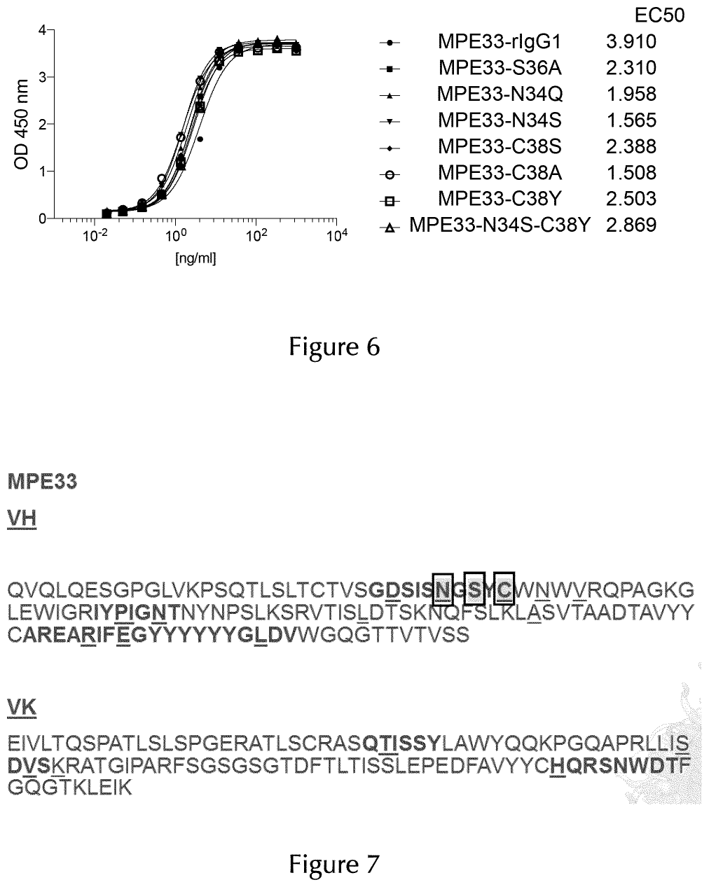

Results are shown in FIG. 6. All tested antibodies MPE33, MPE33_S36A, MPE33_N34Q, MPE33_N34S, MPE33_C38S, MPE33_C38A, MPE33_C38Y and MPE33_N34S_C38Y specifically bound to MPV pre-fusion F protein with similarly high binding affinities. The binding affinities of all variant antibodies were even slightly higher than the binding affinity of MPE33 for MPV pre-fusion F protein, indicating that the various CDRH1 mutations did not impair binding affinity of MPE33 to MPV pre-fusion F protein.

### Example 5: Binding to Cell-Associated F Antigen

Next, binding of all exemplified antibodies of the present invention as described in the above examples as well as of comparative antibody MPE8 was tested.

To this end, Expi293 cells were transfected with MPV F-protein (MPV_NL/1/99_F0-TM (AY304361) and HMPV_Yokohama/JPN(P8527)2016). HMPV_Yokohama/JPN(P8527)2016 carries D280N mutation. Briefly, 10 μg of plasmid DNA were diluted in 0.5 mL of Opti-Mem I medium (Gibco, cat. #31985-047), added to 0.5 mL Opti-Mem containing 30 μl PEI Max transfection reagent (40 kD, cat. # POL24765-1, Polysciences) and incubated 20 min at room temperature (RT). Transfection mix was then added to a culture flask containing 80 ml of expression medium (GIBCO, cat# A14351-02) with growing Expi293 cells (3×10⁶ cells/mL) and further incubated 3 days at 37° C. under agitation. Cells were then harvested, fixed in 4% formaldehyde 20 min on ice, permeabilized with 0.5% Saponin, 1% FBS in PBS 20 min on ice and stained with antibodies MPE33, MPE33_S36A, MPE33_N34Q, MPE33_N34S, MPE33_C38S, MPE33_C38A, MPE33_C38Y and MPE33_N34S_C38Y, MPF5_VH117D (MPF5), MPF5_VH117H and comparative antibody MPE8 (5 μg/ml, 60 min at 4° C. in permeabilization buffer). Binding was revealed by staining cells with AF 647 Goat Anti-Human IgG, Fcγ Fragment Specific secondary antibody (Jackson, 109-606-098, 1 μg/ml 30 min on ice) and by acquiring cells with a flow cytometer.

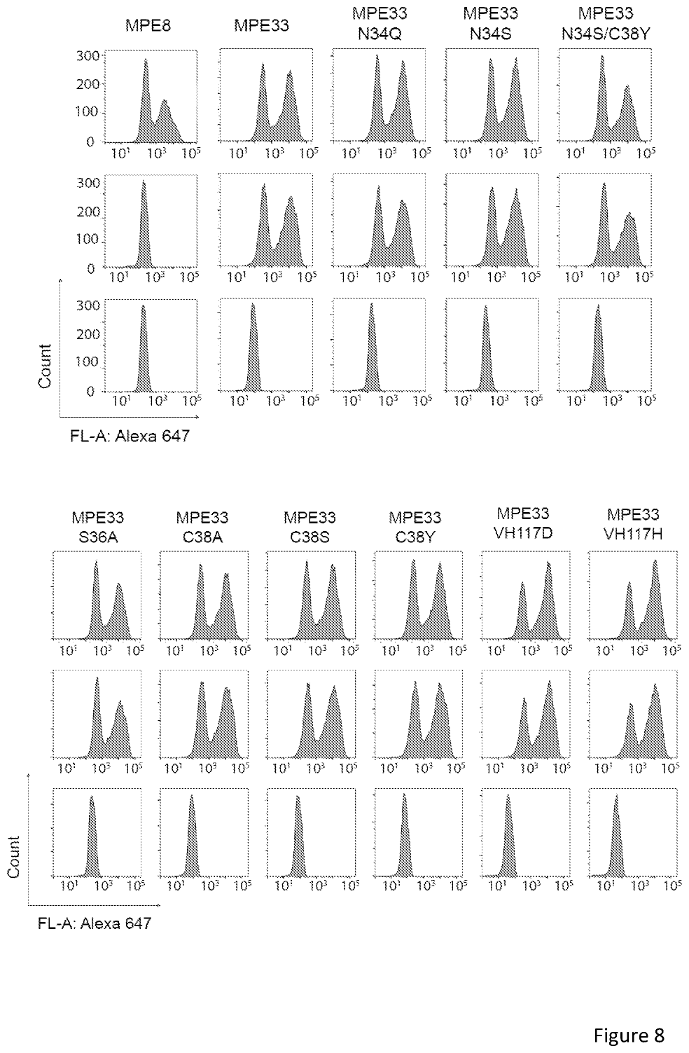

Results are shown in FIG. 8 with MPV_NL/1/99_F0-TM in the upper line, HMPV_Yokohama/JPN(P8527)2016 in the middle line and mock transfection in the lower line. These data show that MPE33 and MPF5 as well as all their variants bind equally well to the F protein of both B1 (NL1/1/99) and B2 (Yokohama) hMPV strains, while the B2 strain (which carries the D280N mutation) results in a viral escape variant for the MPE8 antibody.

### Example 6: Competition Study of Antibodies MPE33, MPF5 and MPE8

To identify whether antibodies MPE33 and MPF5 bind to the same or distinct epitopes on MPV F-protein as comparative antibody MPE8, a binding/competition study was performed.

To this end, competition of antibodies MPE33, MPF5 and MPE8 was assessed by bio-layer interferometry using OCTET RED96 (ForteBio). Briefly, APS sensors were loaded/coated with MPV F-protein at 5 μg/ml in PBS for 10 minutes. Successively sensors were blocked using BSA 1 mg/ml in PBS (blocking buffer) for 5 minutes. Association of the mAbs was performed by moving the sensor into two consecutive wells (7 minutes each) containing the first and second mAb at 30 ug/ml in blocking buffer respectively. All steps were performed at 30° C. under constant mixing at 1000 rpm.

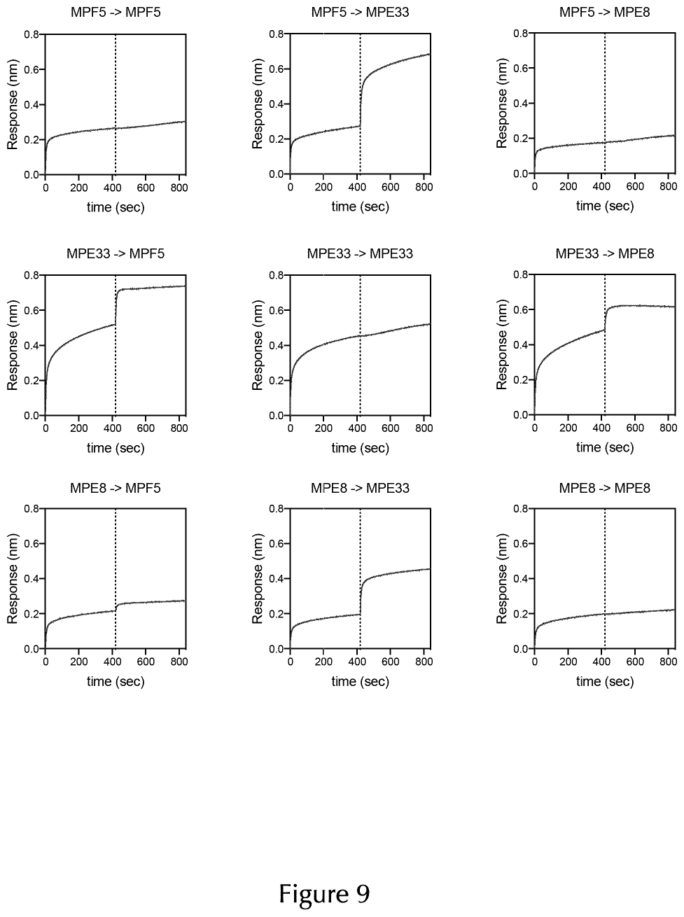

Results are shown in FIG. 9. The data demonstrate that MPF5 competes with MPE8, indicating that both antibodies bind to the same or an overlapping epitope on MPV F-protein. MPE33, however, competes neither with MPF5 nor with MPE8, indicating that MPE33 binds to a distinct epitope on MPV F-protein as compared to MPF5 and MPE8.

TABLE OF SEQUENCES AND SEQ ID NUMBERS (SEQUENCE LISTING):

| | | |
|---|---|---|
| SEQ ID NO | Sequence | Remarks |

Amino acid sequences
MPF5_VH117D

| | | |
|---|---|---|
| SEQ ID NO: 1 | SVSFNDYY | CDRH1 |
| SEQ ID NO: 2 | IGHGGEH | CDRH2 |
| SEQ ID NO: 3 | ARGIGWLPPPD | CDRH3 |
| SEQ ID NO: 4 | QSVLFSSNNENY | CDRL1 |
| SEQ ID NO: 5 | WAS | CDRL2 |
| SEQ ID NO: 6 | LIYWASTRE | CDRL2 long |
| SEQ ID NO: 7 | QQFYSPPWT | CDRL3 |
| SEQ ID NO: 8 | QVQLQQWGAGLLKPSETLSLTCVGNSVSFNDY | VH |
| | YWSWIRQSPGKGLEWIGEIGHGGEHNYNPSLN | |
| | GRVTMSVDTSNNHFSLLLSSVTAADTAMYYCA | |
| | RGIGWLPPPDWGQGTLVTVSS | |
| SEQ ID NO: 9 | DIVMTQSPDSLAVSLGERATINCKSSQSVLFSS | VL |
| | NNENYLAWFQQKPGQPPKLLIYWASTRESGVPD | |
| | RFSGSGSGTDFTLTITSLQAEDVAVYYCQQFYS | |
| | PPWTFGQGTKVEIK | |

MPF5_VH117H

| | | |
|---|---|---|
| SEQ ID NO: 10 | ARGIGWLPPPH | CDRH3 |
| SEQ ID NO: 11 | QVQLQQWGAGLLKPSETLSLTCVGNSVSFNDY | VH |
| | YWSWIRQSPGKGLEWIGEIGHGGEHNYNPSLN | |
| | GRVTMSVDTSNNHFSLLLSSVTAADTAMYYCA | |
| | RGIGWLPPPHWGQGTLVTVSS | |

MPE33

| | | |
|---|---|---|
| SEQ ID NO: 12 | GDSISNGSYC | CDRH1 |
| SEQ ID NO: 13 | TYPIGNT | CDRH2 |
| SEQ ID NO: 14 | AREARIFEGYYYYYYGLDV | CDRH3 |
| SEQ ID NO: 15 | QTISSY | CDRL1 |
| SEQ ID NO: 16 | DVS | CDRL2 |
| SEQ ID NO: 17 | LISDVSKRA | CDRL2 long |
| SEQ ID NO: 18 | HQRSNWDT | CDRL3 |
| SEQ ID NO: 19 | QVQLQESGPGLVKPSQTLSLTCTVSGDSISNGSY | VH |
| | CWNWVRQPAGKGLEWIGRIYPIGNTNYNPSLKSR | |
| | VTISLDTSKNQFSLKLASVTAADTAVYYCAREAR | |
| | IFEGYYYYYYGLDVWGQGTTVTVSS | |
| SEQ ID NO: 20 | EIVLTQSPATLSLSPGERATLSCRASQTISSYLAW | VL |
| | YQQKPGQAPRLLISDVSKRATGIPARFSGSGSGTD | |
| | FTLTISSLEPEDFAVYYCHQRSNWDTFGQGTKLE | |
| | IK | |

MPE33_S36A

| | | |
|---|---|---|
| SEQ ID NO: 21 | GDSISNGAYC | CDRH1 |
| SEQ ID NO: 22 | QVQLQESGPGLVKPSQTLSLTCTVSGDSISNGA | VH |
| | YCWNWVRQPAGKGLEWIGRIYPIGNTNYNPSLK | |
| | SRVTISLDTSKNQFSLKLASVTAADTAVYYCAR | |
| | EARIFEGYYYYYYGLDVWGQGTTVTVSS | |

MPE33_N34Q

| | | |
|---|---|---|
| SEQ ID NO: 23 | GDSISQGSYC | CDRH1 |
| SEQ ID NO: 24 | QVQLQESGPGLVKPSQTLSLTCTVSGDSISQGS | VH |
| | YCWNWVRQPAGKGLEWIGRIYPIGNTNYNPSLK | |
| | SRVTISLDTSKNQFSLKLASVTAADTAVYYCAR | |
| | EARIFEGYYYYYYGLDVWGQGTTVTVSS | |

MPE33_N34S

| | | |
|---|---|---|
| SEQ ID NO: 25 | GDSISSGSYC | CDRH1 |
| SEQ ID NO: 26 | QVQLQESGPGLVKPSQTLSLTCTVSGDSISSGS | VH |
| | YCWNWVRQPAGKGLEWIGRIYPIGNTNYNPSLK | |
| | SRVTISLDTSKNQFSLKLASVTAADTAVYYCAR | |
| | EARIFEGYYYYYYGLDVWGQGTTVTVSS | |

MPE33_C38S

| | | |
|---|---|---|
| SEQ ID NO: 27 | GDSISNGSYS | CDRH1 |
| SEQ ID NO: 28 | QVQLQESGPGLVKPSQTLSLTCTVSGDSISNGS | VH |
| | YSWNWVRQPAGKGLEWIGRIYPIGNTNYNPSLK | |
| | SRVTISLDTSKNQFSLKLASVTAADTAVYYCAR | |
| | EARIFEGYYYYYYGLDVWGOGTTVTVSS | |

MPE33_C38A

| | | |
|---|---|---|
| SEQ ID NO: 29 | GDSISNGSYA | CDRH1 |
| SEQ ID NO: 30 | QVQLQESGPGLVKPSQTLSLTCTVSGDSISNGS | VH |
| | YAWNWVRQPAGKGLEWIGRIYPIGNTNYNPSLK | |
| | SRVTISLDTSKNQFSLKLASVTAADTAVYYCAR | |
| | EARIFEGYYYYYYGLDVWGQGTTVTVSS | |

MPE33_C38Y

| | | |
|---|---|---|
| SEQ ID NO: 31 | GDSISNGSYY | CDRH1 |
| SEQ ID NO: 32 | QVQLQESGPGLVKPSQTLSLTCTVSGDSISNGS | VH |
| | YYWNWVRQPAGKGLEWIGRIYPIGNTNYNPSLK | |
| | SRVTISLDTSKNQFSLKLASVTAADTAVYYCAR | |
| | EARIFEGYYYYYYGLDVWGQGTTVTVSS | |

MPE33_N34S_C38Y

| | | |
|---|---|---|
| SEQ ID NO: 33 | GDSISSGSYY | CDRH1 |
| SEQ ID NO: 34 | QVQLQESGPGLVKPSQTLSLTCTVSGDSISSGS | VH |
| | YYWNWVRQPAGKGLEWIGRIYPIGNTNYNPSLK | |
| | SRVTISLDTSKNQFSLKLASVTAADTAVYYCAR | |
| | EARIFEGYYYYYYGLDVWGQGTTVTVSS | |

Constant regions

| | | |
|---|---|---|
| SEQ ID NO: 35 | ASTKGPSVFPLAPSSKSTSGGTAALGCLVKDYFPE | heavy chain |
| | PVTVSWNSGALTSGVHTFPAVLQSSGLYSLSSVV | |
| | TVPSSSLGTQTYICNVNHKPSNTKVDKKVEPKSC | |
| | DKTHTCPPCPAPELLGGPSVFLFPPKPKDTLMIS | |
| | RTPEVTCVVVDVSHEDPEVKFNWYVDGVEVHNA | |
| | KTKPREEQYNSTYRVVSVLTVLHQDWLNGKEYK | |
| | CKVSNKALPAPIEKTISKAKGQPREPQVYTLPP | |
| | SRDELTKNQVSLTCLVKGFYPSDIAVEWESNGQ | |
| | PENNYKTTPPVLDSDGSFFLYSKLTVDKSRWQQ | |
| | GNVFSCSVLHEALHSHYTQKSLSLSPGK | |
| SEQ ID NO: 36 | GQPKAAPSVTLFPPSSEELQANKATLVCLISDF | lambda light chain |
| | YPGAVTVAWKADSSPVKAGVETTTPSKQSNNKY | |
| | AASSYLSLTPEQWKSHRSYSCQVTHEGSTVEKT | |
| | VAPTECS | |
| SEQ ID NO: 37 | RTVAAPSVFIFPPSDEQLKSGTASVVCLLNNFY | kappa light chain |
| | PREAKVQWKVDNALQSGNSQESVTEQDSKDSTY | |
| | SLSSTLTLSKADYEKHKVYACEVTHQGLSSPVT | |
| | KSFNRGEC | |

Nucleic acid sequences
MPF5_VH117D

| | | |
|---|---|---|
| SEQ ID NO: 38 | AGTGTGTCCTTCAATGATTACTAC | CDRH1 |
| SEQ ID NO: 39 | ATCGGTCACGGTGGAGAACAC | CDRH2 |
| SEQ ID NO: 40 | GCGAGAGGTATTGGCTGGCTCCCCCCTCCG | CDRH3 |
| | GAC | |
| SEQ ID NO: 41 | CAGAGTGTTTTATTCAGCTCCAACAATGAGAA | CDRL1 |
| | CTAC | |
| SEQ ID NO: 42 | TGGGCATCT | CDRL2 |
| SEQ ID NO: 43 | CTCATTTACTGGGCATCTACCCGGGAA | CDRL2 long |
| SEQ ID NO: 44 | CAGCAATTTTATAGTCCTCCGTGGACG | CDRL3 |
| SEQ ID NO: 45 | CAGGTGCAGCTTCAGCAGTGGGGCGCAGGA | VH |
| | CTGTTGAAGCCTTCGGAGACCCTGTCCCTCA | |
| | CCTGCGTTGGAAATAGTGTGTCCTTCAATGAT | |
| | TACTACTGGAGCTGGATCCGCCAGTCCCCAG | |
| | GGAAGGGGCTGGAGTGGATTGGGGAAATC | |
| | GGTCACGGTGGAGAACACAACTACAACCCGT | |
| | CCCTGAACGGTCGAGTCACCATGTCGGTGGAC | |
| | ACGTCCAACAACCATTTCTCCCTACTTCTAT | |
| | CTTCTGTGACCGCCGCGGACACGGCTATGTA | |
| | TTACTGTGCGAGAGGTATTGGCTGGCTCCCC | |
| | CCTCCGGACTGGGGCCAGGGAACCCTGGTC | |
| | ACCGTCTCATCAG | |
| SEQ ID NO: 46 | GACATCGTCATGACTCAGTCTCCAGACTCCC | VL |
| | TGGCTGTGTCTCTGGGCGAGAGGGCCACCAT | |
| | CAACTGCAAGTCCAGCCAGAGTGTTTTATTCA | |
| | GCTCCAACAATGAGAACTACTTAGCTTGGTTC | |
| | CAGCAGAAACCAGGACAGCCTCCTAAGCTAC | |
| | TCATTTACTGGGCATCTACCCGGGAATCCGG | |
| | GGTCCCTGACCGATTCAGTGGCAGCGGGTC | |
| | TGGGACAGATTTCACTCTCACCATCACCAGC | |
| | CTGCAGGCTGAAGATGTGGCAGTTTATTACT | |
| | GTCAGCAATTTTATAGTCCTCCGTGGACGTTC | |
| | GGCCAAGGGACCAAGGTGGAAATCAAAC | |

MPE33

| | | |
|---|---|---|
| SEQ ID NO: 47 | GGTGACTCCATCAGCAACGGTAGTTACTGC | CDRH1 |
| SEQ ID NO: 48 | ATCTATCCCATTGGAAACACC | CDRH2 |
| SEQ ID NO: 49 | GCGAGAGAGGCGAGGATCTTTGAAGGCTAT | CDRH3 |
| | TACTACTACTATTACGGTTTGGACGTC | |
| SEQ ID NO: 50 | CAGACTATAAGTAGTTAC | CDRL1 |
| SEQ ID NO: 51 | GATGTATCC | CDRL2 |
| SEQ ID NO: 52 | CTCATCTCTGATGTATCCAAAAGGGCC | CDRL2 long |
| SEQ ID NO: 53 | CACCAACGTAGCAACTGGGACACT | CDRL3 |
| SEQ ID NO: 54 | CAGGTGCAGCTGCAGGAGTCGGGCCCAGG | VH |
| | ACTGGTGAAGCCTTCACAGACCCTGTCCCTC | |
| | ACCTGCACTGTCTCTGGTGACTCCATCAGCA | |
| | ACGGTAGTTACTGCTGGAATTGGGTCCGGCA | |
| | GCCCGCCGGGAAGGGACTGGAGTGGATTG | |
| | GGCGTATCTATCCCATTGGAAACACCAACTA | |
| | CAACCCCTCCCTCAAGAGTCGAGTCACCATA | |
| | TCACTAGACACGTCCAAGAACCAGTTCTCCC | |
| | TGAAGCTGGCTTCTGTGACCGCCGCAGACAC | |
| | GGCCGTCTACTACTGTGCGAGAGAGGCGAGG | |
| | ATCTTTGAAGGCTATTACTACTACTATTACG | |
| | GTTTGGACGTCTGGGGCCAAGGGACCACGG | |
| | TCACCGTCTCCTCAG | |
| SEQ ID NO: 55 | GAAATTGTGTTGACACAGTCTCCAGCCACCC | VL |
| | TGTCTTTGTCTCCAGGGGAAAGAGCCACCCT | |
| | CTCCTGCAGGGCCAGTCAGACTATAAGTAGT | |
| | TACTTAGCCTGGTACCAACAGAAACCTGGCC | |
| | AGGCTCCCAGGCTCCTCATCTCTGATGTATC | |
| | CAAAAGGGCCACTGGCATCCCAGCCAGGTTC | |
| | AGTGGCAGTGGGTCTGGGACAGACTTCACT | |
| | CTCACCATCAGCAGCCTAGAGCCTGAAGATT | |
| | TTGCAGTTTATTACTGTCACCAACGTAGCAA | |
| | CTGGGACACTTTTGGCCAGGGGACTAAGCTG | |
| | GAGATCAAAC | |

1. An antibody, or an antigen-binding fragment thereof, which binds to the F-protein of metapneumovirus (MPV).

2. The antibody, or an antigen-binding fragment thereof, according to claim 1, wherein the antibody or the antigen-binding fragment thereof comprises (i) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 70% sequence identity with the amino acid sequences of SEQ ID NO: 1, SEQ ID NO: 2, and SEQ ID NO: 3, respectively, and light chain CDR1, CDR2, and CDR3 sequences having at least 70% sequence identity with the amino acid sequences of SEQ ID NO: 4, SEQ ID NO: 6, and SEQ ID NO: 7, respectively; or (iii) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 16, and SEQ ID NO: 18, respectively; or (iv) heavy chain CDR1, CDR2, and CDR3 sequences having at least 70% sequence identity with the amino acid sequences of SEQ ID NO: 12, SEQ ID NO: 13, and SEQ ID NO: 14, respectively, and light chain CDR1, CDR2, and CDR3 sequences having at least 70% sequence identity with the amino acid sequences of SEQ ID NO: 15, SEQ ID NO: 17, and SEQ ID NO: 18, respectively.

3. The antibody, or an antigen-binding fragment thereof, according to any one of the previous claims, wherein the antibody or the antigen-binding fragment thereof binds to the pre-fusion F protein of MPV.

4. The antibody, or an antigen-binding fragment thereof, according to claim 3, wherein an at least 100 fold higher concentration of the antibody, or the antigen-binding fragment thereof, is required for 50% antibody binding to post-fusion F protein of MPV than for 50% antibody binding to pre-fusion F protein of MPV.

5. The antibody, or an antigen-binding fragment thereof, according to any one of the previous claims, wherein the antibody or the antigen-binding fragment thereof neutralizes infection of MPV.

6. The antibody, or an antigen-binding fragment thereof, according to any one of the previous claims, wherein the antibody or the antigen-binding fragment thereof binds specifically to F-proteins of MPV subgroups A1, A2, B1, and B2.

7. The antibody, or an antigen-binding fragment thereof, according to any one of the previous claims, wherein the antibody or the antigen-binding fragment thereof neutralizes infection of MPV subgroups A1, A2, B1, and B2.

8. The antibody, or an antigen-binding fragment thereof, according to any one of the previous claims, wherein the antibody or the antigen-binding fragment thereof comprises (i) heavy chain CDR1, CDR2, and CDR3 sequences having at least 80% sequence identity with the amino acid sequences of SEQ ID NO: 1, SEQ ID NO: 2, and SEQ ID NO: 3, respectively, and light chain CDR1, CDR2, and CDR3 sequences having at least 80% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 80% sequence identity with the amino acid sequences of SEQ ID NO: 1, SEQ ID NO: 2, and SEQ ID NO: 3, respectively, and light chain CDR1, CDR2, and CDR3 sequences having at least 80% sequence identity with the amino acid sequences of SEQ ID NO: 4, SEQ ID NO: 6, and SEQ ID NO: 7, respectively; or (iii) heavy chain CDR1, CDR2, and CDR3 sequences having at least 80% sequence identity with the amino acid sequences of SEQ ID NO: 12, SEQ ID NO: 13, and SEQ ID NO: 14, respectively, and light chain CDR1, CDR2, and CDR3 sequences having at least 80% sequence identity with the amino acid sequences of SEQ ID NO: 15, SEQ ID NO: 16, and SEQ ID NO: 18, respectively; or (iv) heavy chain CDR1, CDR2, and CDR3 sequences having at least 80% sequence identity with the amino acid sequences of SEQ ID NO: 12, SEQ ID NO: 13, and SEQ ID NO: 14, respectively, and light chain CDR1, CDR2, and CDR3 sequences having at least 80% sequence identity with the amino acid sequences of SEQ ID NO: 15, SEQ ID NO: 17, and SEQ ID NO: 18, respectively.

9. The antibody, or an antigen-binding fragment thereof, according to any one of the previous claims, wherein the antibody or the antigen-binding fragment thereof comprises (i) heavy chain CDR1, CDR2, and CDR3 sequences having at least 90% sequence identity with the amino acid sequences of SEQ ID NO: 1, SEQ ID NO: 2, and SEQ ID NO: 3, respectively, and light chain CDR1, CDR2, and CDR3 sequences having at least 90% sequence identity with the amino acid sequences of SEQ ID NO: 4, SEQ ID NO: 5, and SEQ ID NO: 7, respectively; or (ii) heavy chain CDR1, CDR2, and CDR3 sequences having at least 90% sequence identity with the amino acid sequences of SEQ ID NO: 1, SEQ ID NO: 2, and SEQ ID NO: 3, respectively, and light chain CDR1, CDR2, and CDR3 sequences having at least 90% sequence identity with the amino acid sequences of SEQ ID NO: 4, SEQ ID NO: 6, and SEQ ID NO: 7, respectively; or (iii) heavy chain CDR1, CDR2, and CDR3 sequences having at least 90% sequence identity with the amino acid sequences of SEQ ID NO: 12, SEQ ID NO: 13, and SEQ ID NO: 14, respectively, and light chain CDR1, CDR2, and CDR3 sequences having at least 90% sequence identity with the amino acid sequences of SEQ ID NO: 15, SEQ ID NO: 16, and SEQ ID NO: 18, respectively; or (iv) heavy chain CDR1, CDR2, and CDR3 sequences having at least 90% sequence identity with the amino acid sequences of SEQ ID NO: 12, SEQ ID NO: 13, and SEQ ID NO: 14, respectively, and light chain CDR1, CDR2, and CDR3 sequences having at least 90% sequence identity with the amino acid sequences of SEQ ID NO: 15, SEQ ID NO: 17, and SEQ ID NO: 18, respectively.

10. The antibody, or an antigen-binding fragment thereof, according to any one of the previous claims, wherein the antibody or the antigen-binding fragment thereof comprises:
a heavy chain CDR1 sequence according to SEQ ID NO: 1;
a heavy chain CDR2 sequence according to SEQ ID NO: 2;
a heavy chain CDR3 sequence having at least 90% sequence identity with the amino acid sequences of SEQ ID NO: 3;
a light chain CDR1 sequence according to SEQ ID NO: 4;
a light chain CDR2 sequence according to SEQ ID NO: 5 or 6; and
a light chain CDR3 sequence according to SEQ ID NO: 7.

11. The antibody, or an antigen-binding fragment thereof, according to claim 10, wherein the C-terminal Asp residue in SEQ ID NO: 3 is substituted; optionally with another polar amino acid.

12. The antibody, or an antigen-binding fragment thereof, according to claim 10 or 11, wherein the antibody or the antigen-binding fragment thereof comprises a heavy chain CDR3 sequence according to SEQ ID NO: 3 or 10.

13. The antibody, or an antigen-binding fragment thereof, according to any one of the previous claims, wherein the antibody or the antigen-binding fragment thereof comprises:
a heavy chain CDR1 sequence according to SEQ ID NO: 1;
a heavy chain CDR2 sequence according to SEQ ID NO: 2;
a heavy chain CDR3 sequence according to SEQ ID NO: 3;
a light chain CDR1 sequence according to SEQ ID NO: 4;
a light chain CDR2 sequence according to SEQ ID NO: 5 or 6; and
a light chain CDR3 sequence according to SEQ ID NO: 7.

14. The antibody, or an antigen-binding fragment thereof, according to any one of the previous claims, wherein the antibody or the antigen-binding fragment thereof comprises:
a heavy chain CDR1 sequence according to SEQ ID NO: 1;
a heavy chain CDR2 sequence according to SEQ ID NO: 2;
a heavy chain CDR3 sequence according to SEQ ID NO: 10;
a light chain CDR1 sequence according to SEQ ID NO: 4;
a light chain CDR2 sequence according to SEQ ID NO: 5 or 6; and
a light chain CDR3 sequence according to SEQ ID NO: 7.

15. The antibody, or an antigen-binding fragment thereof, according to any one of claims 1 to 9, wherein the antibody or the antigen-binding fragment thereof comprises:
a heavy chain CDR1 sequence having at least 70% sequence identity with the amino acid sequences of SEQ ID NO: 12;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

16. The antibody, or an antigen-binding fragment thereof, according to claim 15, wherein one or more of the heavy chain variable region amino acid residues N34, S36 and C38 (corresponding to N6, S8 and C10, respectively, in SEQ ID NO: 12) is/are substituted.

17. The antibody, or an antigen-binding fragment thereof, according to claim 16, wherein
N34 (corresponding to N6 in SEQ ID NO: 12) is substituted with Gln (Q) or Ser (S);
S36 (corresponding to S8 in SEQ ID NO: 12) is substituted with Ala (A); and/or
C38 (corresponding to C10 in SEQ ID NO: 12) is substituted with Ser (S), Ala (A) or Tyr (Y).

18. The antibody, or an antigen-binding fragment thereof, according to any one of claims 15 to 17, wherein the heavy chain CDR1 sequence differs in a single or exactly two amino acid substitution(s) from SEQ ID NO: 12.

19. The antibody, or an antigen-binding fragment thereof, according to any one of claims 15 to 18, wherein the antibody, or an antigen-binding fragment thereof, comprises a heavy chain CDR1 sequence according to any one of SEQ ID NOs 12, 21, 23, 25, 27, 29, 31 and 33.

20. The antibody, or an antigen-binding fragment thereof, according to any one of claims 15 to 19, wherein the antibody or the antigen-binding fragment thereof comprises:
a heavy chain CDR1 sequence according to SEQ ID NO: 12;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

21. The antibody, or an antigen-binding fragment thereof, according to any one of claims 15 to 19, wherein the antibody or the antigen-binding fragment thereof comprises:
a heavy chain CDR1 sequence according to SEQ ID NO: 21;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

22. The antibody, or an antigen-binding fragment thereof, according to any one of claims 15 to 19, wherein the antibody or the antigen-binding fragment thereof comprises:
a heavy chain CDR1 sequence according to SEQ ID NO: 23;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

23. The antibody, or an antigen-binding fragment thereof, according to any one of claims 15 to 19, wherein the antibody or the antigen-binding fragment thereof comprises:
a heavy chain CDR1 sequence according to SEQ ID NO: 25;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

24. The antibody, or an antigen-binding fragment thereof, according to any one of claims 15 to 19, wherein the antibody or the antigen-binding fragment thereof comprises:
a heavy chain CDR1 sequence according to SEQ ID NO: 27;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

25. The antibody, or an antigen-binding fragment thereof, according to any one of claims 15 to 19, wherein the antibody or the antigen-binding fragment thereof comprises:
a heavy chain CDR1 sequence according to SEQ ID NO: 29;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

26. The antibody, or an antigen-binding fragment thereof, according to any one of claims 15 to 19, wherein the antibody or the antigen-binding fragment thereof comprises:
a heavy chain CDR1 sequence according to SEQ ID NO: 31;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

27. The antibody, or an antigen-binding fragment thereof, according to any one of claims 15 to 19, wherein the antibody or the antigen-binding fragment thereof comprises:
a heavy chain CDR1 sequence according to SEQ ID NO: 33;
a heavy chain CDR2 sequence according to SEQ ID NO: 13;
a heavy chain CDR3 sequence according to SEQ ID NO: 14;
a light chain CDR1 sequence according to SEQ ID NO: 15;
a light chain CDR2 sequence according to SEQ ID NO: 16 or 17; and
a light chain CDR3 sequence according to SEQ ID NO: 18.

28. The antibody, or an antigen-binding fragment thereof, according to any one of the previous claims, wherein the antibody or the antigen-binding fragment thereof comprises (i) a heavy chain variable region comprising an amino acid sequence having at least 70% identity to SEQ ID NO: 8 and a light chain variable region comprising the amino acid sequence having at least 70% identity to SEQ ID NO: 9; or (ii) a heavy chain variable region comprising an amino acid sequence having at least 70% identity to SEQ ID NO: 19 and a light chain variable region comprising the amino acid sequence having at least 70% identity to SEQ ID NO: 20.

29. The antibody, or an antigen-binding fragment thereof, according to any one of the previous claims, wherein the antibody or the antigen-binding fragment thereof comprises (i) a heavy chain variable region comprising an amino acid sequence having at least 75% identity to SEQ ID NO: 8 and a light chain variable region comprising the amino acid sequence having at least 75% identity to SEQ ID NO: 9; or (ii) a heavy chain variable region comprising an amino acid sequence having at least 75% identity to SEQ ID NO: 19 and a light chain variable region comprising the amino acid sequence having at least 75% identity to SEQ ID NO: 20.

30. The antibody, or an antigen-binding fragment thereof, according to any one of the previous claims, wherein the antibody or the antigen-binding fragment thereof comprises (i) a heavy chain variable region comprising an amino acid sequence having at least 80% identity to SEQ ID NO: 8 and a light chain variable region comprising the amino acid sequence having at least 80% identity to SEQ ID NO: 9; or (ii) a heavy chain variable region comprising an amino acid sequence having at least 80% identity to SEQ ID NO: 19 and a light chain variable region comprising the amino acid sequence having at least 80% identity to SEQ ID NO: 20.

31. The antibody, or an antigen-binding fragment thereof, according to any one of the previous claims, wherein the antibody or the antigen-binding fragment thereof comprises (i) a heavy chain variable region comprising an amino acid sequence having at least 85% identity to SEQ ID NO: 8 and a light chain variable region comprising the amino acid sequence having at least 85% identity to SEQ ID NO: 9; or (ii) a heavy chain variable region comprising an amino acid sequence having at least 85% identity to SEQ ID NO: 19 and a light chain variable region comprising the amino acid sequence having at least 85% identity to SEQ ID NO: 20.

32. The antibody, or an antigen-binding fragment thereof, according to any one of the previous claims, wherein the antibody or the antigen-binding fragment thereof comprises (i) a heavy chain variable region comprising an amino acid sequence having at least 90% identity to SEQ ID NO: 8 and a light chain variable region comprising the amino acid sequence having at least 90% identity to SEQ ID NO: 9; or (ii) a heavy chain variable region comprising an amino acid sequence having at least 90% identity to SEQ ID NO: 19 and a light chain variable region comprising the amino acid sequence having at least 90% identity to SEQ ID NO: 20.

33. The antibody, or an antigen-binding fragment thereof, according to any one of the previous claims, wherein the antibody or the antigen-binding fragment thereof comprises (i) a heavy chain variable region comprising an amino acid sequence having at least 95% identity to SEQ ID NO: 8 and a light chain variable region comprising the amino acid sequence having at least 95% identity to SEQ ID NO: 9; or (ii) a heavy chain variable region comprising an amino acid sequence having at least 95% identity to SEQ ID NO: 19 and a light chain variable region comprising the amino acid sequence having at least 95% identity to SEQ ID NO: 20.

34. The antibody, or an antigen-binding fragment thereof, according to any one of the previous claims, wherein the antibody or the antigen-binding fragment thereof comprises a heavy chain variable region comprising an amino acid sequence according to SEQ ID NO: 8 and a light chain variable region according to SEQ ID NO: 9.

35. The antibody, or an antigen-binding fragment thereof, according to any one of the previous claims, wherein the antibody or the antigen-binding fragment thereof comprises a heavy chain variable region comprising an amino acid sequence according to SEQ ID NO: 11 and a light chain variable region according to SEQ ID NO: 9.

36. The antibody, or an antigen-binding fragment thereof, according to any one of the previous claims, wherein the antibody or the antigen-binding fragment thereof comprises a heavy chain variable region comprising an amino acid sequence according to SEQ ID NO: 19 and a light chain variable region according to SEQ ID NO: 20.

37. The antibody, or an antigen-binding fragment thereof, according to any one of the previous claims, wherein the antibody or the antigen-binding fragment thereof comprises a heavy chain variable region comprising an amino acid sequence according to SEQ ID NO: 22 and a light chain variable region according to SEQ ID NO: 20.

38. The antibody, or an antigen-binding fragment thereof, according to any one of the previous claims, wherein the antibody or the antigen-binding fragment thereof comprises a heavy chain variable region comprising an amino acid sequence according to SEQ ID NO: 24 and a light chain variable region according to SEQ ID NO: 20.

39. The antibody, or an antigen-binding fragment thereof, according to any one of the previous claims, wherein the antibody or the antigen-binding fragment thereof comprises a heavy chain variable region comprising an amino acid sequence according to SEQ ID NO: 26 and a light chain variable region according to SEQ ID NO: 20.

40. The antibody, or an antigen-binding fragment thereof, according to any one of the previous claims, wherein the antibody or the antigen-binding fragment thereof comprises a heavy chain variable region comprising an amino acid sequence according to SEQ ID NO: 28 and a light chain variable region according to SEQ ID NO: 20.

41. The antibody, or an antigen-binding fragment thereof, according to any one of the previous claims, wherein the antibody or the antigen-binding fragment thereof comprises a heavy chain variable region comprising an amino acid sequence according to SEQ ID NO: 30 and a light chain variable region according to SEQ ID NO: 20.

42. The antibody, or an antigen-binding fragment thereof, according to any one of the previous claims, wherein the antibody or the antigen-binding fragment thereof comprises a heavy chain variable region comprising an amino acid sequence according to SEQ ID NO: 32 and a light chain variable region according to SEQ ID NO: 20.

43. The antibody, or an antigen-binding fragment thereof, according to any one of the previous claims, wherein the antibody or the antigen-binding fragment thereof comprises a heavy chain variable region comprising an amino acid sequence according to SEQ ID NO: 34 and a light chain variable region according to SEQ ID NO: 20.

44. The antibody, or an antigen-binding fragment thereof, according to any one of the previous claims, wherein the antibody or the antigen-binding fragment thereof is a human antibody.

45. The antibody, or an antigen-binding fragment thereof, of any one of the previous claims, wherein the antibody, or an antigen-binding fragment thereof, is a monoclonal antibody.

46. The antibody of any one of the previous claims, wherein the antibody comprises an Fc moiety.

47. The antibody of any one of the previous claims, wherein the antibody is of the IgG type.

48. The antibody of claim 47, wherein the antibody is of the IgG1 type.

49. The antibody, or an antigen-binding fragment thereof, of any one of the previous claims, wherein the antibody, or the antigen-binding fragment thereof, is purified.

50. The antibody, or an antigen-binding fragment thereof, of any one of the previous claims, wherein the antibody, or the antigen-binding fragment thereof, is a single-chain antibody.

51. The antibody, or an antigen-binding fragment thereof, of any one of the previous claims, wherein the antibody, or the antigen-binding fragment thereof, is selected from Fab, Fab′, F(ab′)2, Fv or scFv.

52. The antibody, or an antigen-binding fragment thereof, of any one of the previous claims for use as a medicament.

53. The antibody, or an antigen-binding fragment thereof, for use according to claim 52 in prophylaxis or treatment of MPV infection.

54. A nucleic acid molecule comprising a polynucleotide encoding the antibody, or an antigen-binding fragment thereof, of any one of claims 1 to 51.

55. The nucleic acid molecule of claim 54, wherein the polynucleotide encoding the antibody, or an antigen-binding fragment thereof, is codon-optimized.

56. The nucleic acid molecule of claim 54 or 55 comprising a nucleic acid sequence as set forth in any one of SEQ ID NOs 38-55; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

57. A combination of a first and a second nucleic acid molecule, wherein the first nucleic acid molecule comprises a polynucleotide encoding the heavy chain of the antibody, or an antigen-binding fragment thereof, of any one of claims 1 to 51; and the second nucleic acid molecule comprises a polynucleotide encoding the corresponding light chain of the same antibody, or the same antigen-binding fragment thereof.

58. The combination of nucleic acid molecules of claim 57, wherein one or both of the polynucleotides encoding the heavy and/or light chain(s) of the antibody, or an antigen-binding fragment thereof, is/are codon-optimized.

59. The combination of nucleic acid molecules of claim 57 or 58 comprising a nucleic acid sequence as set forth in any one of SEQ ID NOs 38-55; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

60. A combination of a first and a second nucleic acid molecule, wherein
(i) the first nucleic acid molecule comprises a polynucleotide encoding the heavy chain of an antibody, or an antigen-binding fragment thereof, the polynucleotide comprising: (a) nucleotide sequences according to SEQ ID NOs 38, 39 and 40; or (b) nucleotide sequences according to SEQ ID NOs 47, 48 and 49; and
(ii) the second nucleic acid molecule comprises a polynucleotide encoding the light chain of an antibody, or an antigen-binding fragment thereof, the polynucleotide comprising: (c) nucleotide sequences according to SEQ ID NOs 41, 42 (or 43) and 44; or (d) nucleotide sequences according to SEQ ID NOs 50, 51 (or 52) and

53.

61. A vector comprising the nucleic acid molecule of any one of claims 54 to 56 or the combination of nucleic acid molecules of any one of claims 57 to 60.

62. A combination of a first and a second vector, wherein the first vector comprises a first nucleic acid molecule as defined in any one of claims 57 to 60 and the second vector comprises the corresponding second nucleic acid molecule as defined in any one of claims 57 to 60.

63. A cell expressing the antibody, or an antigen-binding fragment thereof, of any one of claims 1 to 51, or comprising the vector of claim 61 or the combination of vectors of claim 62.

64. A pharmaceutical composition comprising the antibody, or an antigen-binding fragment thereof, of any one of claims 1 to 51, the nucleic acid of any one of claims 54 to 56, the combination of nucleic acids of any one of claims 57 to 60, the vector of claim 61, the combination of vectors of claim 62 or the cell of claim 63, and, optionally, a pharmaceutically acceptable excipient, diluent or carrier.

65. The antibody, or an antigen-binding fragment thereof, of any one of claims 1 to 51, the nucleic acid of any one of claims 54 to 56, the combination of nucleic acids of any one of claims 57 to 60, the vector of claim 61, the combination of vectors of claim 62, the cell of claim 63, or the pharmaceutical composition of claim 64 for use as a medicament; optionally in the prophylaxis or treatment of MPV infection.

66. Use of the antibody, or an antigen-binding fragment thereof, of any one of claims 1 to 51 in (in-vitro) diagnosis of MPV infection.

67. Use of the antibody, or an antigen-binding fragment thereof, of any one of claims 1 to 51 in a method for detecting MPV antigens.

68. Use of the antibody, or an antigen-binding fragment thereof, of any one of claims 1 to 51 for monitoring the quality of anti-MPV vaccines by checking the antigen of said vaccine.

69. The use according to claim 68, wherein the conformation of the antigen, or an epitope thereof, of said vaccine is checked.

70. Use of the antibody, or an antigen-binding fragment thereof, of any one of claims 1 to 51, the nucleic acid of any one of claims 54 to 56, the combination of nucleic acids of any one of claims 57 to 60, the vector of claim 61, the combination of vectors of claim 62, the cell of claim 63, or the pharmaceutical composition of claim 64 in the manufacture of a medicament for prophylaxis, treatment or attenuation of MPV infection.

71. A method of reducing MPV infection, or lowering the risk of MPV infection, comprising: administering to a subject in need thereof, a therapeutically effective amount of the antibody, or an antigen-binding fragment thereof, of any one of claims 1 to 51, the nucleic acid of any one of claims 54 to 56, the combination of nucleic acids of any one of claims 57 to 60, the vector of claim 61, the combination of vectors of claim 62, the cell of claim 63, or the pharmaceutical composition of claim 64.

72. A method for testing an anti-MPV vaccine, wherein the vaccine is contacted with the antibody, or an antigen-binding fragment thereof, of any one of claims 1 to 51 and, optionally, the presence of antibody/antigen complexes is determined.


SEQUENCE LISTING (55 sequences)

SEQ ID NO: 1 - protein, 8 aa, Artificial Sequence
  MPF5_VH117D CDRH1
SVSFNDYY

SEQ ID NO: 2 - protein, 7 aa, Artificial Sequence
  MPF5_VH117D CDRH2
IGHGGEH

SEQ ID NO: 3 - protein, 11 aa, Artificial Sequence
  MPF5_VH117D CDRH3
ARGIGWLPPPD

SEQ ID NO: 4 - protein, 12 aa, Artificial Sequence
  MPF5_VH117D CDRL1
QSVLFSSNNENY

SEQ ID NO: 5 - skipped

SEQ ID NO: 6 - protein, 9 aa, Artificial Sequence
  MPF5_VH117D CDRL2 long
LIYWASTRE

SEQ ID NO: 7 - protein, 9 aa, Artificial Sequence
  MPF5_VH117D CDRL3
QQFYSPPWT

SEQ ID NO: 8 - protein, 117 aa, Artificial Sequence
  MPF5_VH117D VH
QVQLQQWGAGLLKPSETLSLTCVGNSVSFNDYYWSWIRQSPGKGLEWIGEIGHGGEHNYNPSLNGRVTMSVDTSNNHFSLLLSSVTAADTAMYYCARGIGWLPPPDWGQGTLVTVSS

SEQ ID NO: 9 - protein, 113 aa, Artificial Sequence
  MPF5_VH117D VL
DIVMTQSPDSLAVSLGERATINCKSSQSVLFSSNNENYLAWFQQKPGQPPKLLIYWASTRESGVPDRFSGSGSGTDFTLTITSLQAEDVAVYYCQQFYSPPWTFGQGTKVEIK

SEQ ID NO: 10 - protein, 11 aa, Artificial Sequence
  MPF5_VH117H CDRH3
ARGIGWLPPPH

SEQ ID NO: 11 - protein, 117 aa, Artificial Sequence
  MPF5_VH117H VH
QVQLQQWGAGLLKPSETLSLTCVGNSVSFNDYYWSWIRQSPGKGLEWIGEIGHGGEHNYNPSLNGRVTMSVDTSNNHFSLLLSSVTAADTAMYYCARGIGWLPPPHWGQGTLVTVSS

SEQ ID NO: 12 - protein, 10 aa, Artificial Sequence
  MPE33 CDRH1
GDSISNGSYC

SEQ ID NO: 13 - protein, 7 aa, Artificial Sequence
  MPE33 CDRH2
IYPIGNT

SEQ ID NO: 14 - protein, 19 aa, Artificial Sequence
  MPE33 CDRH3
AREARIFEGYYYYYYGLDV

SEQ ID NO: 15 - protein, 6 aa, Artificial Sequence
  MPE33 CDRL1
QTISSY

SEQ ID NO: 16 - skipped

SEQ ID NO: 17 - protein, 9 aa, Artificial Sequence
  MPE33 CDRL2 long
LISDVSKRA

SEQ ID NO: 18 - protein, 8 aa, Artificial Sequence
  MPE33 CDRL3
HQRSNWDT

SEQ ID NO: 19 - protein, 127 aa, Artificial Sequence
  MPE33 VH
QVQLQESGPGLVKPSQTLSLTCTVSGDSISNGSYCWNWVRQPAGKGLEWIGRIYPIGNTNYNPSLKSRVTISLDTSKNQFSLKLASVTAADTAVYYCAREARIFEGYYYYYYGLDVWGQGTTVTVSS

SEQ ID NO: 20 - protein, 106 aa, Artificial Sequence
  MPE33 VL
EIVLTQSPATLSLSPGERATLSCRASQTISSYLAWYQQKPGQAPRLLISDVSKRATGIPARFSGSGSGTDFTLTISSLEPEDFAVYYCHQRSNWDTFGQGTKLEIK

SEQ ID NO: 21 - protein, 10 aa, Artificial Sequence
  MPE33_S36A CDRH1
GDSISNGAYC

SEQ ID NO: 22 - protein, 127 aa, Artificial Sequence
  MPE33_S36A VH
QVQLQESGPGLVKPSQTLSLTCTVSGDSISNGAYCWNWVRQPAGKGLEWIGRIYPIGNTNYNPSLKSRVTISLDTSKNQFSLKLASVTAADTAVYYCAREARIFEGYYYYYYGLDVWGQGTTVTVSS

SEQ ID NO: 23 - protein, 10 aa, Artificial Sequence
  MPE33_N34Q CDRH1
GDSISQGSYC

SEQ ID NO: 24 - protein, 127 aa, Artificial Sequence
  MPE33_N34Q VH
QVQLQESGPGLVKPSQTLSLTCTVSGDSISQGSYCWNWVRQPAGKGLEWIGRIYPIGNTNYNPSLKSRVTISLDTSKNQFSLKLASVTAADTAVYYCAREARIFEGYYYYYYGLDVWGQGTTVTVSS

SEQ ID NO: 25 - protein, 10 aa, Artificial Sequence
  MPE33_N34S CDRH1
GDSISSGSYC

SEQ ID NO: 26 - protein, 127 aa, Artificial Sequence
  MPE33_N34S VH
QVQLQESGPGLVKPSQTLSLTCTVSGDSISSGSYCWNWVRQPAGKGLEWIGRIYPIGNTNYNPSLKSRVTISLDTSKNQFSLKLASVTAADTAVYYCAREARIFEGYYYYYYGLDVWGQGTTVTVSS

SEQ ID NO: 27 - protein, 10 aa, Artificial Sequence
  MPE33_C38S CDRH1
GDSISNGSYS

SEQ ID NO: 28 - protein, 127 aa, Artificial Sequence
  MPE33_C38S VH
QVQLQESGPGLVKPSQTLSLTCTVSGDSISNGSYSWNWVRQPAGKGLEWIGRIYPIGNTNYNPSLKSRVTISLDTSKNQFSLKLASVTAADTAVYYCAREARIFEGYYYYYYGLDVWGQGTTVTVSS

SEQ ID NO: 29 - protein, 10 aa, Artificial Sequence
  MPE33_C38A CDRH1
GDSISNGSYA

SEQ ID NO: 30 - protein, 127 aa, Artificial Sequence
  MPE33_C38A VH
QVQLQESGPGLVKPSQTLSLTCTVSGDSISNGSYAWNWVRQPAGKGLEWIGRIYPIGNTNYNPSLKSRVTISLDTSKNQFSLKLASVTAADTAVYYCAREARIFEGYYYYYYGLDVWGQGTTVTVSS

SEQ ID NO: 31 - protein, 10 aa, Artificial Sequence
  MPE33_C38Y CDRH1
GDSISNGSYY

SEQ ID NO: 32 - protein, 127 aa, Artificial Sequence
  MPE33_C38Y VH
QVQLQESGPGLVKPSQTLSLTCTVSGDSISNGSYYWNWVRQPAGKGLEWIGRIYPIGNTNYNPSLKSRVTISLDTSKNQFSLKLASVTAADTAVYYCAREARIFEGYYYYYYGLDVWGQGTTVTVSS

SEQ ID NO: 33 - protein, 10 aa, Artificial Sequence
  MPE33_N34S_C38Y CDRH1
GDSISSGSYY

SEQ ID NO: 34 - protein, 127 aa, Artificial Sequence
  MPE33_N34S_C38Y VH
QVQLQESGPGLVKPSQTLSLTCTVSGDSISSGSYYWNWVRQPAGKGLEWIGRIYPIGNTNYNPSLKSRVTISLDTSKNQFSLKLASVTAADTAVYYCAREARIFEGYYYYYYGLDVWGQGTTVTVSS

SEQ ID NO: 35 - protein, 330 aa, Artificial Sequence
  heavy chain constant regions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 36 - protein, 106 aa, Artificial Sequence
  lambda light chain constant region
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 107 aa, Artificial Sequence [sequence omitted]

SEQ ID NO: 38 - DNA, 24 bp, Artificial Sequence
  MPF5_VH117D CDRH1
agtgtgtccttcaatgattactac

SEQ ID NO: 39 - DNA, 21 bp, Artificial Sequence
  MPF5_VH117D CDRH2
atcggtcacggtggagaacac

SEQ ID NO: 40 - DNA, 33 bp, Artificial Sequence
  MPF5_VH117D CDRH3
gcgagaggtattggctggctcccccctccggac

SEQ ID NO: 41 - DNA, 36 bp, Artificial Sequence
  MPF5_VH117D CDRL1
cagagtgttttattcagctccaacaatgagaactac

SEQ ID NO: 42 - skipped

SEQ ID NO: 43 - DNA, 27 bp, Artificial Sequence
  MPF5_VH117D CDRL2 long
ctcatttactgggcatctacccgggaa

SEQ ID NO: 44 - DNA, 27 bp, Artificial Sequence
  MPF5_VH117D CDRL3
cagcaattttatagtcctccgtggacg

SEQ ID NO: 45 - DNA, 352 bp, Artificial Sequence
  MPF5_VH117D VH
caggtgcagcttcagcagtggggcgcaggactgttgaagccttcggagaccctgtccctcacctgcgttggaaatagtgtgtccttcaatgattactactggagctggatccgccagtccccagggaaggggctggagtggattggggaaatcggtcacggtggagaacacaactacaacccgtccctgaacggtcgagtcaccatgtcggtggacacgtccaacaaccatttctccctacttctatcttctgtgaccgccgcggacacggctatgtattactgtgcgagaggtattggctggctcccccctccggactggggccagggaaccctggtcaccgtctcatcag

SEQ ID NO: 46 - DNA, 340 bp, Artificial Sequence
  MPF5_VH117D VL
gacatcgtcatgactcagtctccagactccctggctgtgtctctgggcgagagggccaccatcaactgcaagtccagccagagtgttttattcagctccaacaatgagaactacttagcttggttccagcagaaaccaggacagcctcctaagctactcatttactgggcatctacccgggaatccggggtccctgaccgattcagtggcagcgggtctgggacagatttcactctcaccatcaccagcctgcaggctgaagatgtggcagtttattactgtcagcaattttatagtcctccgtggacgttcggccaagggaccaaggtggaaatcaaac

SEQ ID NO: 47 - DNA, 30 bp, Artificial Sequence
  MPE33 CDRH1
ggtgactccatcagcaacggtagttactgc

SEQ ID NO: 48 - DNA, 21 bp, Artificial Sequence
  MPE33 CDRH2
atctatcccattggaaacacc

SEQ ID NO: 49 - DNA, 57 bp, Artificial Sequence
  MPE33 CDRH3
gcgagagaggcgaggatctttgaaggctattactactactattacggtttggacgtc

SEQ ID NO: 50 - DNA, 18 bp, Artificial Sequence
  MPE33 CDRL1
cagactataagtagttac

SEQ ID NO: 51 - skipped

SEQ ID NO: 52 - DNA, 27 bp, Artificial Sequence
  MPE33 CDRL2 long
ctcatctctgatgtatccaaaagggcc

SEQ ID NO: 53 - DNA, 24 bp, Artificial Sequence
  MPE33 CDRL3
caccaacgtagcaactgggacact

SEQ ID NO: 54 - DNA, 382 bp, Artificial Sequence
  MPE33 VH
caggtgcagctgcaggagtcgggcccaggactggtgaagccttcacagaccctgtccctcacctgcactgtctctggtgactccatcagcaacggtagttactgctggaattgggtccggcagcccgccgggaagggactggagtggattgggcgtatctatcccattggaaacaccaactacaacccctccctcaagagtcgagtcaccatatcactagacacgtccaagaaccagttctccctgaagctggcttctgtgaccgccgcagacacggccgtctactactgtgcgagagaggcgaggatctttgaaggctattactactactattacggtttggacgtctggggccaagggaccacggtcaccgtctcctcag

SEQ ID NO: 55 - DNA, 319 bp, Artificial Sequence
  MPE33 VL
gaaattgtgttgacacagtctccagccaccctgtctttgtctccaggggaaagagccaccctctcctgcagggccagtcagactataagtagttacttagcctggtaccaacagaaacctggccaggctcccaggctcctcatctctgatgtatccaaaagggccactggcatcccagccaggttcagtggcagtgggtctgggacagacttcactctcaccatcagcagcctagagcctgaagattttgcagtttattactgtcaccaacgtagcaactgggacacttttggccaggggactaagctggagatcaaac